(19) United States
(12) Patent Application Publication
(10) Pub. No.: US 2010/0209399 A1
(43) Pub. Date: Aug. 19, 2010

(54) BRAIN-DERIVED STEM CELLS FOR REPAIR OF MUSCULOSKELETAL SYSTEM IN VERTEBRATE SUBJECTS

(75) Inventor: Oleg V. Kopyov, Moorpark, CA (US)

Correspondence Address: KAREN S. CANADY;CANADY & LORTZ LLP, 4201 Wilshire BLV Suite 622, LOS ANGELES, CA, 90010, US

(73) Assignee: Celavie Biosciences, LLC, Reading, PA (US)

(21) Appl. No.: 12/578,263

(22) Filed: Oct. 13, 2009

Related U.S. Application Data
(60) Provisional application No. 61/152,498, filed on Feb. 13, 2009.

(51) Int. Cl. A61K 35/12

(52) U.S. Cl. 424/93.7

(57) ABSTRACT

The stem cells that can be propagated and maintained for extended periods of time in culture in the absence of a feeder layer, and can be used to repair tissue damage. These cells are derived from fetal tissues and are able to repair different types of damage in musculoskeletal system, with significantly greater efficacy than stem cells derived from adult tissues. These cells are hypoimmunogenic and can be used for allogeneic transplantation to vertebrate hosts having disease and/or damage in musculoskeletal and other tissues. The cells can be administered by direct injection to the site in need of repair or by systemic (e.g., intravenous) administration. The stem cells of the invention are capable of migrating to the sites in need of repair, and of adopting a phenotype most appropriate to the nature of the damage, injury or disease.

This application claims the benefit of provisional patent application No. 61/152,498, filed Feb. 13, 2009, the entire contents of which are incorporated herein by reference.

This application is related to U.S. patent application Ser. No. 11/755,224, filed May 30, 2007, which application claims the benefit of provisional patent application No. 60/803,619, filed May 31, 2006; and is a continuation-in-part of U.S. patent application Ser. No. 11/002,933, filed Dec. 2, 2004, which claims priority to provisional application No. 60/526,242, filed Dec. 2, 2003, the entire contents of each of which are incorporated by reference herein. Throughout this application various publications are referenced. The disclosures of these publications in their entireties are hereby incorporated by reference into this application in order to describe more fully the state of the art to which this invention pertains.

### BACKGROUND OF THE INVENTION

Standard treatments for musculoskeletal injury (MSI), such as anti-inflammatory medication, bracing, rest and physical therapy, is insufficient in many cases. Previous studies using adipose and bone marrow derived adult stem cells for the treatment of tendinitis in experimental studies in the horse show a modest improvement in tendon repair (Schnabel et al. 2007; Nixon et al. 2008). Numerous other mechanisms including growth factor injections, pharmaceutical injections, and surgical procedures, have been shown to minimally improve the recovery after tendonitis in horses.

Some veterinarians use autologous fat or bone marrow-derived nucleated cells. This approach involves aspiration of fat or bone-marrow from the injured recipient horse, a procedure that is burdened by pain and significant risk of infection. Adult stem cells represent multipotent cells that have the capability of differentiating into various types of connective tissues. The origin of multipotent stem cells in adults can vary, with most currently being derived from the bone marrow, umbilical perivascular tissues, blood, muscle, and more recently adipose tissue. Adult stem cells represent multipotent cells that have the capability of differentiating into various types of connective tissues. The origin of multipotent stem cells in adults can vary, with most currently being derived from the bone marrow, perivascular tissues, blood, muscle, and more recently adipose tissue. All can be used as a source of autogenous multipotent cell for transplantation. However, many of these autogenous techniques rely on tissue harvest, a protracted culture phase, and occasionally cell sorting, to develop a uniform pool of graftable cells. Moreover, controlled studies suggest neither marrow derived nor adipose derived stem cell therapies make a dramatic difference to accelerate equine tendon healing (Schnabel et al. 2007; Nixon et al. 2008).

On the other hand, allogeneic fetal-derived stem cells show much higher restorative potential in a variety of tissues and organs in multiple species. They can be procured from any mare, be expanded indefinitely and stored in liquid nitrogen banks without significant loss of viability and restorative potential. Moreover, allogeneic embryonic and fetal-derived stem cells (ESC) do not express proteins that expose foreign substances to the host immune system and do not elicit immune response, thereby eliminating the necessity of immune suppression.

Tendon and ligament injuries are the bane of the performance horse industry. These injuries cause loss of performance and decrease in value to the equine. Conventional therapies include confinement, anti-inflammatory medications and bandaging. The chance for possible re-injury is greater when the injury heals with malaligned fibers and scar tissue. The ideal is for the tendon and ligament to heal with a minimum amount of scar tissue.

There remains a need for more effective therapies for repair of connective tissue and other musculoskeletal tissues.

### SUMMARY OF THE INVENTION

The invention provides stem cells that can be propagated and maintained for extended periods of time in culture in the absence of a feeder layer, and that can be used to repair tissue damage. Although these cells are derived from different fetal tissues (brain, heart, liver, etc.), they are able to repair injured or diseased tissues of the musculoskeletal system as well as the central nervous system of vertebrate subjects with significantly greater efficacy than stem cells derived from adult tissues. These cells are hypoimmunogenic, as they do not express MHC, and can be used for allogeneic transplantation to vertebrate hosts having disease and/or damage in musculoskeletal, central nervous system (CNS), and other tissues. The ability to repair damage has been documented for the musculoskeletal system of competitive and companion horses and dogs, and can be adapted to cats and other species. The ability to repair damage has also been documented for the CNS of rodent subjects transplanted with human stem cells, and has been extended to canine subjects. Once injected in the injured site, the stem cells of the invention differentiate according to a phenotype most appropriate to the nature of the injured part of the musculoskeletal system and restore normal or near normal structure of the injured or diseased tissue. Moreover, the stem cells of the invention have been found to have an ability to protect against massive structural and functional damage.

The pluripotent nature of these cells renders it unnecessary to genetically modify the cells to be transplanted, and also obviates concerns about selecting the appropriate phenotype of cells, or predifferentiating cells prior to transplantation. Accordingly, the invention provides, in one embodiment, a substantially pure culture of pluripotent cells that is free of genetically modified cells. Use of these pluripotent cells provides particular advantages for transplantation and therapy over, for example, use of predifferentiated cells. The cells of the invention also offer advantages over blastocyst-derived stem cells, as the cells of the invention do not form tumors nor do they show signs of developing mutations or karyotypic abnormalities, even after more than six months in culture.

The invention provides a method of ameliorating tissue injury in a vertebrate subject by introducing into a site of tissue injury in the subject at least 0.5-1 million stem cells, wherein the stem cells are derived from fetal mammalian tissues (brain, heart, liver, etc.). A variety of tissues can be treated with these cells, including musculoskeletal tissues. Also provided is a method of repairing diseased or injured connective tissue, and a method of treating diseased or injured connective tissue. Each method comprises the step of introducing into the site of disease or injury, at least 0.5-1 million stem cells.

Typically, the vertebrate subject is a mammal, and includes human, equine, canine, feline, ovine, porcine, bovine and other veterinary subjects. The tissue damage includes damage due to disease or injury. In a typical embodiment, the tissue is musculoskeletal tissue, such as connective tissue, joint tissue, muscle or bone. In one embodiment, the introducing is by injection into the site of damage. The injection can be performed under ultrasound guidance. In another embodiment, the introducing is by implanting the cells into an area that communicates with the site of injury or disease such that the stem cells arrive at the site of damage by migration or via the circulatory system, such as by intravenous administration. Intravenous administration can be systemic or localized. One example of localized intravenous administration of the cells of the invention is distal end perfusion.

In one embodiment, about 0.5 to about 10 million stem cells are introduced into the site of damage. In a typical embodiment, about 1-1.2 million stem cells are introduced. The stem cells are immunopositive for telomerase, TRA-1-60, TRA-1-81, Oct-4, nestin, SSEA-4 and Nanog, and do not express major histocompatibility complex (MHC) or p53. The cells can therefore be used for allogeneic treatment. The stem cells are typically cultured for at least 30-90 days, prior to the introducing. The stem cells can be cultured in a medium having a total calcium concentration of 0.03 to 0.15 mM and comprising:
(a) about 15-100 ng/ml epidermal growth factor (EGF);
(b) about 10-150 ng/ml basic fibroblast growth factor (bFGF);
(c) about 10-75 ng/ml transforming growth factor-alpha (TGFα); and
(d) about 30-50 ng/ml insulin-like growth factor (IGF).

Optionally, the medium further comprises one or all of the following:
(e) about 1-3% by volume B27;
(f) about 40-60 ng/ml leukemia inhibitory factor (LIF);
(g) about 0.05-0.2 mM GLUTAMAX; and
(h) about 0.5-2% by volume N2 supplement.

In another embodiment, the medium comprises:
(a) about 15-100 ng/ml epidermal growth factor (EGF);
(b) about 10-150 ng/ml basic fibroblast growth factor (bFGF); and
(c) about 10-75 ng/ml transforming growth factor-alpha (TGFα);
(d) about 10-100 ng/ml leukemia inhibiting factor;
(e) about 10-100 ng/ml amphiregulin;
(f) about 10-100 ng of caspase inhibitor;
(g) about 10-100 ng/ml pifithrin.

In a more specific embodiment, the medium is Eagle's minimum essential medium (EMEM) and comprises:
(a) about 40 ng/ml epidermal growth factor (EGF);
(b) about 40 ng/ml basic fibroblast growth factor (bFGF); and
(c) about 40 ng/ml transforming growth factor-alpha (TGFα).
(d) about 40 ng/ml insulin-like growth factor (IGF);
(e) about 50 ng/ml leukemia inhibitory factor (LIF);
(f) about 2% by volume B27;
(g) about 0.05-0.2 mM GLUTAMAX;
(h) about 0.5-2% by volume N2 supplement; and
(i) about 0.05 mM calcium chloride.

The cells can be derived from human, equine, canine or feline fetal brain. The cells can also be derived from other visceral organs, such as heart or liver.

Examples of connective tissue damage include, but are not limited to, ligament injury, osteochondrosis, tendonitis, navicular syndrome, cartilage damage, laminitis or arthritis.

The invention further provides a kit comprising a container, the container comprising one or more doses of about 1 to about 2 million cells each; typically about 1.2 million cells each. A kit may comprise as many as 10 million cells. Typically the cells are in a cryopreservation or culture medium of the invention, in a volume of, for example, about 2 mL. Larger volumes, such as about 60 ml, and larger doses, may be more suitable for intravenous administration. The appropriate number of doses is selected based on the nature, size and severity of injury or disease. The kit further comprises a label that indicates use of the cells for implantation into a site of tissue damage, such as connective tissue damage. Optionally, the kit additionally comprises a needle and/or a syringe suitable for transcutaneous intra-connective tissue or intra-venous injection. In one embodiment, the container comprising the stem cells is a syringe. The syringe can be prepared so that its contents remain aseptic and ready for injection, e.g., by merely attaching a needle to the syringe.

The kit can further comprise a second container, the second container comprising a supplemental composition for introducing into the site of damage together with the stem cells. Examples of supplemental compositions include, but are not limited to platelet-rich plasma, growth factors, and interleukin-1 receptor antagonist protein (IRAP). In one embodiment, the second container is a chamber attached to the first container. For example, where the first container is a syringe, the second container can be attached to the syringe, its contents separated from the contents of the first container by a destructible barrier. Upon breach of the barrier, the contents of the second container enter into the first container and mix with the stem cells, for injection as a single composition. Kits of the invention optionally further comprise instructions for use in accordance with one or more methods of the invention. The instructions can be provided in print form or via other media, including, for example, a computer readable disc, such as a digital video disc, portable drive, memory card, or compact disc.

### BRIEF DESCRIPTION OF THE FIGURES

FIG. 1 is a set of photomicrographs showing cell immunostaining before transplant.

FIG. 2 is a pair of ultrasonographs of treated tendon from a 3 year old quarterhorse mare who presented with an injury to the left front superficial digital flexor tendon. The left panel shows a pre-treatment ultrasound. The right panel depicts an ultrasound taken 5 weeks post-transplant with a dose of 1 million stem cells via injection into the site of injury. The lateral zone 2A-3B shows 90% improvement.

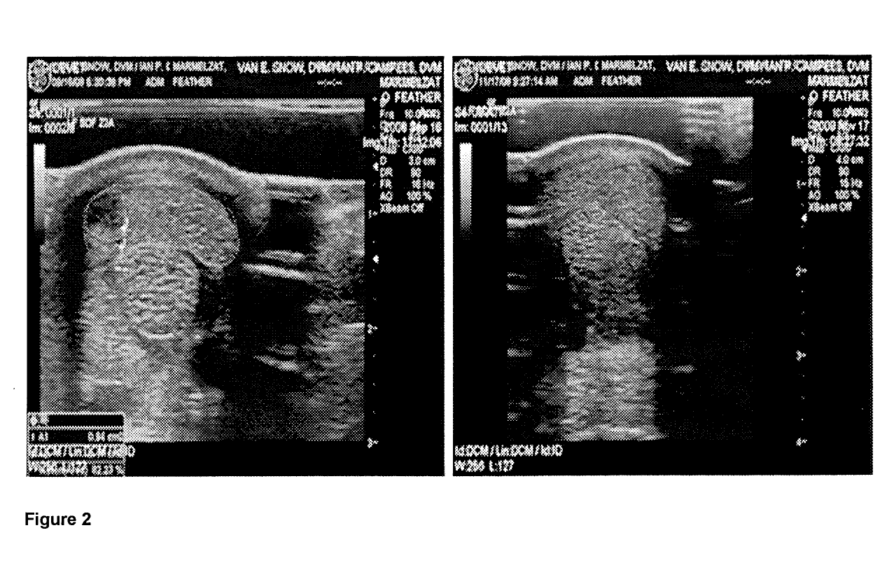

FIG. 3 is a graph depicting the effect on fiber alignment of injection of different doses of allogeneic stem cells into injured tendons over a period of 120 days. The effect is measured by a scoring system of 0-4, with 0 being normal. ‘>1.2M’ indicates doses of 2 and 5 million cells. The graph shows no significant difference between a dose of 1.2 million cells, and ‘>1.2M’.

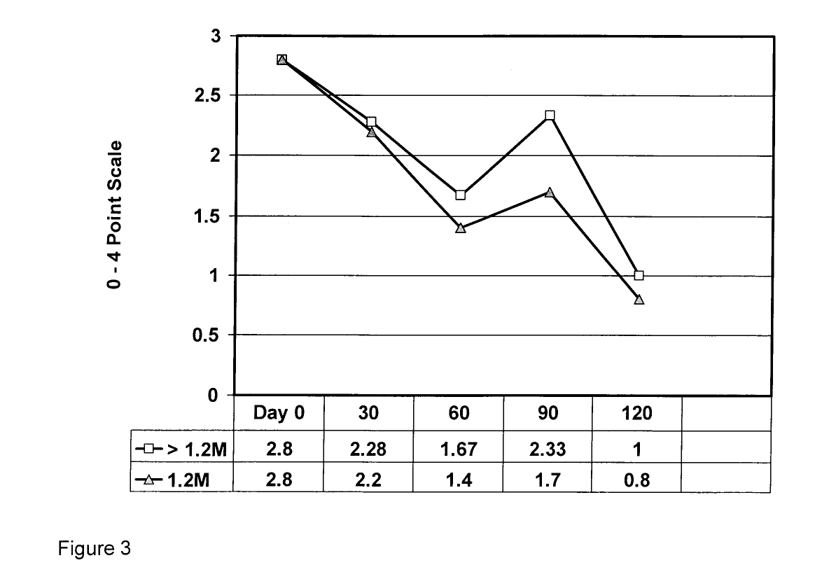

FIG. 4 is a graph depicting the effect on echogenicity of injection of different doses of allogeneic stem cells into injured tendons over a period of 120 days. The effect is measured by a scoring system of 0-4, with 0 being normal. ‘>1.2M’ indicates doses of 2 and 5 million cells. The graph shows no significant difference between a dose of 1.2 million cells, and ‘>1.2M’.

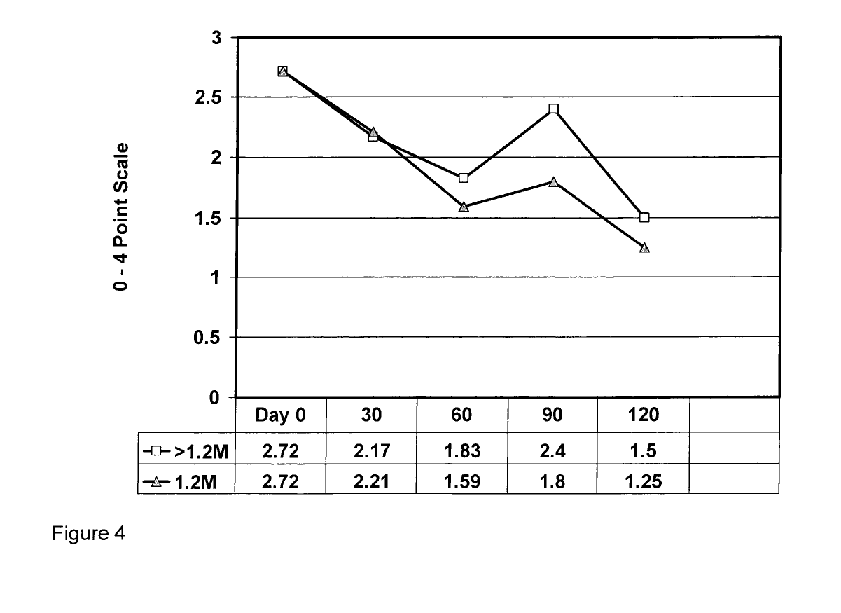

FIG. 5 is a graph depicting the effect on lameness of injection of different doses of allogeneic stem cells into injured tendons over a period of 120 days. The effect is measured by a scoring system of 0-5, with 0 being normal. ‘>1.2M’ indicates doses of 2 and 5 million cells. The graph shows no significant difference between a dose of 1.2 million cells, and ‘>1.2M’.

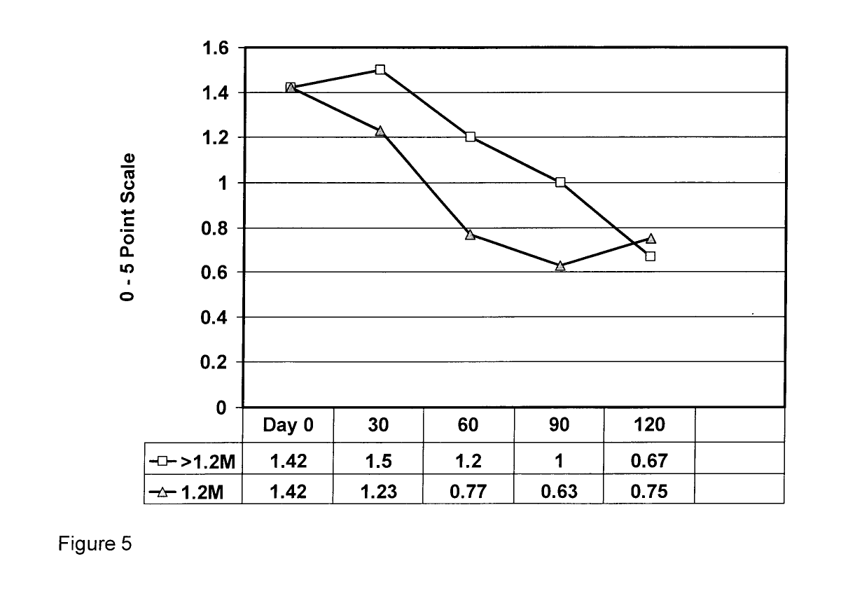

FIG. 6 is a graph depicting the effect on pain after injection of different doses of allogeneic stem cells into injured tendons over a period of 120 days. The effect is measured by a scoring system of 0-1, with 0 being no pain. ‘>1.2M’ indicates doses of 2 and 5 million cells. The graph shows no significant difference between a dose of 1.2 million cells, and ‘>1.2M’.

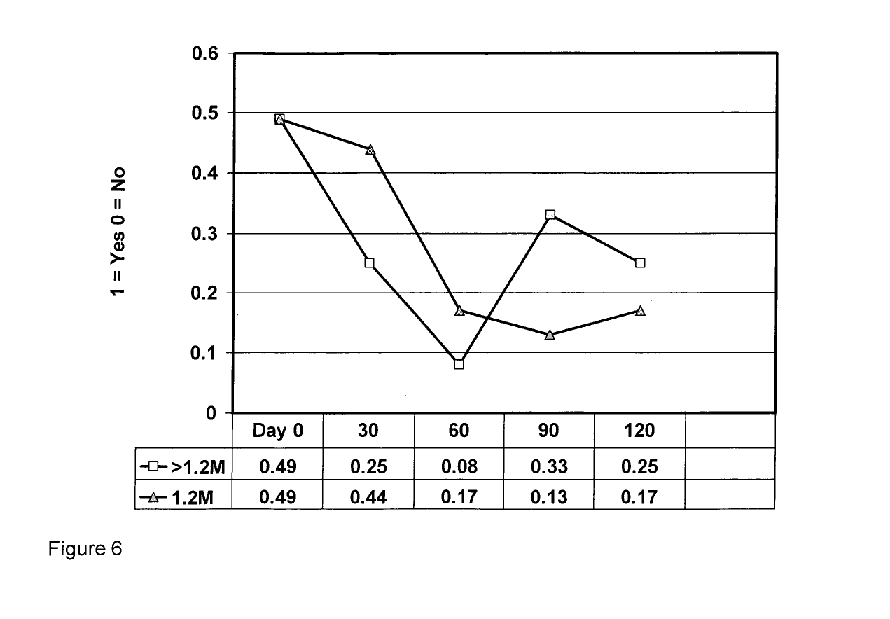

FIG. 7 is a graph depicting the effect on exercise levels after injection of different doses of allogeneic stem cells into injured tendons over a period of 120 days. The effect is measured by a scoring system of 0-7, with 0 being complete stall rest and 7 being maximal exercise levels. ‘>1.2M’ indicates doses of 2 and 5 million cells. The graph shows no significant difference between a dose of 1.2 million cells, and ‘>1.2M’.

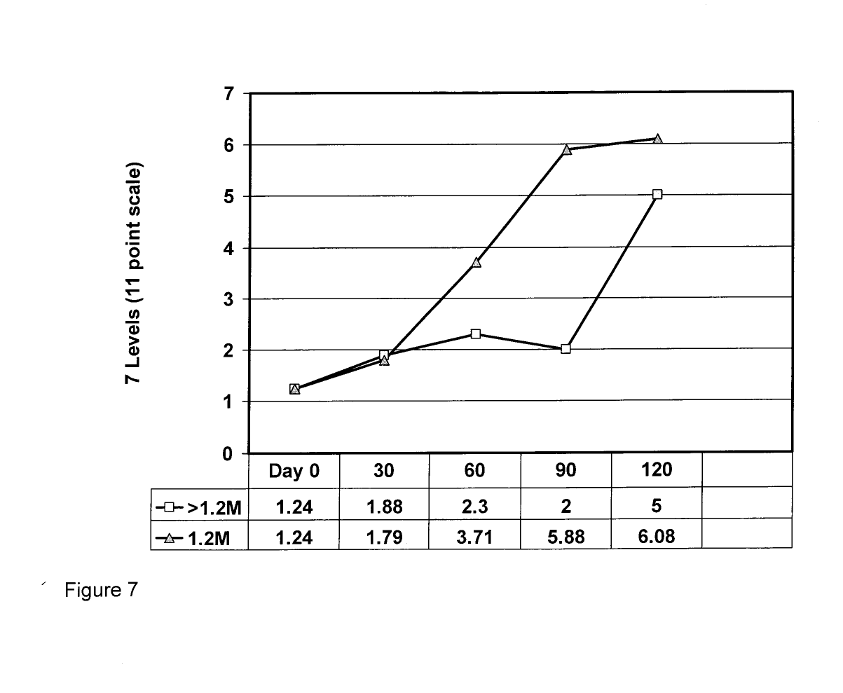

FIG. 8 is a series of sonograms from the ultrasound assessment of a representative subject from the experimental (Horse 6) and control (Horse 5) groups, taken at 0, 2, 4 and 8 weeks following collagenase injection. The tendon is shown in both cross-section (left columns) and longitudinal views (right columns). At 2 weeks, the collagenase-induced lesion is visible in both the control and experimental subjects. By 4 weeks, significant improvement is seen in the experimental subjects, but not in the controls. At 8 weeks, not only is the wound healed, but it exhibits remarkable fiber alignment, in contrast with the disorganized scar tissue observed in the control subject.

FIG. 9 is a series of cross-sectional images of the affected tendon viewed via MRI, which is more sensitive to scar tissue. The top row shows images from each of the experimental subjects taken 8 weeks following collagenase injection. Shown in the lower row are images from each of the control subjects, taken at the same time point. The arrows point to the area of disorganized (scar) tissue, which is significantly more present in control subjects.

FIG. 10 is a set of photomicrographs of histological sections stained with hematoxylin and eosin (left images) and visualized with polarization (right side), showing the very different tendon architecture of experimental tendon (normally-aligned tendon fibers; upper images) versus control tendon (disorganized; lower images).

FIG. 11 is a set of photomicrographs taken with fluorescence to confirm survival of transplanted cells by detecting the presence of the Y chromosome in male cells transplanted into a female host. At two weeks post injection (upper images), a number of rounded cells are positive for the Y chromosome. The boxed area in the upper left image is shown, enlarged, in the upper right image. By 8 weeks (lower images), the Y-positive cells are no longer rounded, but have become aligned tendon fibers.

### DETAILED DESCRIPTION OF THE INVENTION

The present invention is based on the discovery of stem cells derived from brain, heart or liver of vertebrate subjects that can be propagated and maintained for extended periods of time in culture in the absence of a feeder layer. These visceral organ-derived stem cells can be used to repair damage in the musculoskeletal system of vertebrate subjects with significantly greater efficacy than stem cells derived from other tissues, such as skin, cartilage, pancreas and lung. These cells are useful for allogeneic transplantation to hosts having disease and/or damage. The ability to repair damage with allogeneic transplants has been documented for connective tissue. The stem cells of the invention are capable of migrating to the sites in need of repair, and of adopting a phenotype appropriate to the nature of the damage or disease. Moreover, the stem cells of the invention have been found to have a surprising ability to protect against massive structural and functional damage.

Studies described herein have shown embryonic stem cells of the invention to provide an effective alternative regenerative therapy for tendon and ligament injuries. Embryonic derived stem cells are pluripotent and non-immunogenic, which gives them the capability to generate almost any type of cells without the danger of immune mediated rejection. Ultrasonography shows that the treated tendons and ligaments heal with a matrix more like the original tissue and less like scar tissue. Horses with tendonitis and torn ligaments that are treated with embryonic derived stem cells of the invention are able to resume training sooner with a better quality of healing. With more than 60 horses treated with stem cells of the invention, not a single instance of teratoma or tumor formation has been observed, providing a significant advantage over blastocyst-derived stem cells.

### DEFINITIONS

All scientific and technical terms used in this application have meanings commonly used in the art unless otherwise specified. As used in this application, the following words or phrases have the meanings specified.

As used herein, “connective tissue” means what is known as connective tissue proper, including areolar (loose) connective tissue and fibrous connective tissue, such as tendons and ligaments. Also included are specialized connective tissues, such as cartilage and reticular connective tissue.

As used herein, “musculoskeletal system” refers to all components of this system, including muscle, bone, joints and connective tissues.

As used herein, “low calcium” medium refers to less than 0.15 mM calcium (final concentration), and typically about 0.03-0.09 mM. Low calcium medium does not include calcium-free medium. “High calcium” medium refers to greater than 0.15 mM calcium.

As used herein, “pluripotent cell” (PC; or pluripotent stem cell, PSC) or “stem cell” refers to cells that are immunopositive for the pluripotent cell markers, TRA-1-60, TRA-1-81, SSEA-4, Nanog and Oct-4 (transcription factor octamer-4).

As used herein, “fetal”, such as in “fetal-derived stem cell”, refers to what is understood in the art to be fetus, encompassing developing mammalian organisms after the blastocyst stage and prior to birth (e.g., full-term).

As used herein, “genetically modified” refers to cells that have been manipulated to contain a non-native transgene by recombinant methods. For example, cells can be genetically modified by introducing a nucleic acid molecule that encodes a selected polypeptide.

As used herein, “transgene” means DNA that is inserted into a cell and that encodes an amino acid sequence corresponding to a functional protein. Typically, the encoded protein is capable of exerting a therapeutic or regulatory effect.

As used herein, “protein” or “polypeptide” includes proteins, functional fragments of proteins, and peptides, whether isolated from natural sources, produced by recombinant techniques or chemically synthesized. Polypeptides typically comprise at least about 6 amino acids, and are sufficiently long to exert a biological or therapeutic effect.

As used herein, “pharmaceutically acceptable carrier” includes any material which, when combined with an active ingredient, allows the ingredient to retain biological activity and is non-reactive with the subject's immune system. Examples include, but are not limited to, any of the standard pharmaceutical carriers such as a phosphate buffered saline solution, water, emulsions such as oil/water emulsion, and various types of wetting agents. Preferred diluents for parenteral administration of stem cells include phenol red-free Eagle's minimum essential medium (EMEM; Biowhittaker).

Compositions comprising such carriers are formulated by well known conventional methods (see, for example, Remington's Pharmaceutical Sciences, 18th edition, A. Gennaro, ed., Mack Publishing Co., Easton, Pa., 1990).

As used herein, “a” or “an” means at least one, unless clearly indicated otherwise.

### Pluripotent Stem Cells

The invention provides pluripotent stem cells (referred to herein as PSC or PC) that can be maintained indefinitely in culture, stain positively for bromodeoxyuridine (BrdU), TRA-1-60, TRA-1-81, SSEA-4, Oct-4, Nanog and nestin, negative for an apoptotic marker p53 and are pluripotent. PSC of the invention can be maintained in cell culture, typically as a suspension culture, for at least one year. The PC described herein have been maintained for more than two years.

The PSC of the invention exhibit 50% growth in the first 2 days in culture, and doubling times of less than 15 days, typically about 12 days. Doubling times of as little as 5 days have been observed. In addition, these cells continue to grow in culture for extended periods of time. Unlike neural progenitor cells cultured in conventional media such as Neurobasal™ medium, however, these cultures do not show a decline after 3-4 months, but continue to survive and expand for years, and through hundreds of passages.

In addition, the PSC of the invention exhibit normal structure and function that is typical of progenitor cells. All cells show normal karyotype, even when cultured beyond 6 months. PSC form embryoid bodies in culture. PSC can grow in floating clusters, or also form a confluent growth of PSC that remain undifferentiated.

PC can be prepared from fetal brain, as described in Example 1 below. Typically, the tissue (ectodermal tissue that develops into CNS) is dissected in a general purpose serum-free medium, such as Hank's Balanced Salt Solution (HBSS) with 0.25 μg/ml of Fungizone and 10 μg/ml of Gentamicin, under sterile conditions. Dissection of fetal brain tissue from fetuses of differing ages can be guided by anatomical guides known in the art, such as Mosenthal, W. T., 1995, A Textbook of Neuroanatomy: with atlas and dissection guide (Taylor & Francis).

The cultures described herein will initially include a small percentage of Oct-4-, TRA-1-60-, TRA-1-81-, SSEA-4-, and nestin-positive PC cells. Over a period of 1 to 6 months in culture, the proportion of Oct-4-, TRA-1-60-, TRA-1-81-, nestin-, and SSEA-4-positive cells increases significantly. For example, a typical culture will shift from being 5% Oct-4-positive cells to about 30% Oct-4-positive cells within 30 days, to up to 95% Oct-4-positive cells after four months in culture.

The pluripotent nature of these cells makes them attractive for placement in a variety of tissue environments, wherein local cytokines (natural and/or exogenously supplied) and other signals induce appropriate differentiation and migration. The PSC do not express MHC, making them suitable for allogeneic transplants. Typically, the cells are derived from the same species as the recipient.

### Media and Methods for Cell Culture

The structure and function of PC in culture is subject to manipulation via the culture medium. For example, raising the calcium concentration of the medium from 0.05 mM to 0.1 mM leads to attachment of the progenitor cells to the culture flask. The addition of LIF to the culture medium shortens the doubling time and prevents spontaneous differentiation. TGFα, amphiregulin, caspase inhibitor and pifithrin (an inhibitor of p53) in the medium serve to reduce doubling time (e.g., from 15 days to 8 days). Accordingly, the culture medium is selected in accordance with the particular objectives, with some ingredients favoring growth and expansion and other ingredients favoring attachment and differentiation.

For general purposes, the cell culture requires a low calcium basal medium (e.g., Ca++ free EMEM supplemented with calcium chloride), typically a B27, N2 or equivalent supplement, and growth factors (e.g., EGF, FGF, TGFα, amphiregulin). Optional ingredients include L-glutamine or, preferably, GLUTAMAX (Invitrogen, Carlsbad, Calif.), which promotes viability, and LIF that prevents differentiation.

A detailed description of the optimization of culture media for expansion and for differentiation of PC can be found in U.S. patent application Ser. No. 11/002,933, filed Dec. 2, 2004. In general, long-term growth and expansion requires a low calcium concentration. This is typically achieved by use of a calcium-free minimum essential medium (EMEM) or phenol red-free EMEM to which calcium is added. Optimal growth and expansion has been observed at calcium concentrations of 0.05-0.06 mM. As the calcium concentration rises, e.g., above 0.15 mM, network formations between the neurons in culture are observed as they take on a more differentiated neuronal phenotype. In these higher calcium cultures, only 1-2% of the cells are immunopositive for the astrocytic marker GFAP.

The following table summarizes the range of concentrations suitable for culture medium components:

| Component: | Concentration: |
|---|---|
| B27 | 0.5-2.5% |
| Calcium Chloride | 0.05 mM-0.12 mM |
| Epidermal Growth Factor | 15 ng/mL-100 ng/mL |
| Basic Fibroblast Growth Factor | 10 ng/mL-150 ng/mL |
| Transforming Growth Factor Alpha | 10 ng/mL-75 ng/mμL |
| Leukemia Inhibitory Factor | 10 ng/mL-100 ng/mL |
| Glutamax ™ | 0.1 mM-0.7 mM |
| N2 Supplement | 0.3%-2.0% |
| Amphiregulin | 10-100 ng/ml |
| caspase inhibitor | 10-100 ng/ml |
| pifithrin | 10-100 ng/ml |

PSC are typically grown in suspension cultures. Initial plating of primary cells was optimal at 50,000 to 80,000 cells/ml. Medium changes can be made every 6 days (complete feeding) by removing the cells to a test tube and spinning (e.g., 5 min at 1,000 rpm). Typically, all but 2 ml of the supernatant is discarded and the pellet is resuspended in the remaining 2 ml of supernatant combined with an additional 4 ml of fresh medium. Additionally, 3 days after complete feeding 4 ml of fresh medium is added to the flask. When density exceeds 10,000,000 cells/ml, the cells can be split into two or more culture flasks (e.g., T75 flasks). Trituration of the cells at the time of feeding helps to break up clusters of PC and maintain them as a single cell suspension in the culture medium. Those skilled in the art will appreciate that variation of these parameters will be tolerated and can be optimized to suit particular objectives and conditions.

### Cryopreservation of PC

The ability to store and successfully thaw PC and PC is valuable to their utility in clinical applications and ensuring a continued and consistent supply of suitable cells. While most experts working with progenitor and pluripotent cell populations observe only a 2-30% survival of cells after freeze-thaw, the present invention offers media and methods that result in over 70-80% survival following freeze-thaw, with viability typically greater than 85%.

For cryopreservation, PC are suspended in a low calcium medium supplemented with B27, DMSO, MEM non-essential amino acids solution (Gibco, N.Y.) and the trophic factors used in the expansion culture medium. Typically, the growth factors in the cryopreservation medium comprise about 20-100 ng/ml epidermal growth factor (EGF); about 10-50 ng/ml fibroblast growth factor basic (bFGF); and about 1-150 ng/ml transforming growth factor-alpha (TGFα). The cells are placed at −20° C. for 30 min, followed by −70° C. overnight, and then placed in liquid nitrogen.

For thawing, both the culture medium and the flask, or other vessel into which the cells will be grown, are pre-warmed to 15-40° C., preferably to approximately 25-37° C. Typically, culture flasks (or other vessel) are pre-warmed in an incubator with the same or similar gas, humidity and temperature conditions as will be used for growing the cells. For example, typical temperature is about 37° C., and typical CO₂ level is about 8% and O₂ level is about 3%.

### Kits of the Invention

The PC of the invention can be used in therapeutic and diagnostic applications, as well as for drug screening and genetic manipulation. The PC and/or culture media of the invention can be provided in kit form, optionally including containers and/or syringes and other materials, rendering them ready for use in any of these applications. In a typical embodiment, the kit comprises a container comprising one or more doses of about 1 to 2 million, typically 1.2 million, stem cells of the invention. Multi-dose kits can contain multiples of such doses. Such doses can be packaged separately or combined to facilitate multiple serial administrations to more than one site. The kit further comprises a label that indicates use of the cells for implantation into a site of tissue damage, such as connective tissue or other musculoskeletal injury or disease.

Optionally, the kit additionally comprises a needle suitable for intra-connective tissue or intra-venous injection and/or a syringe. In one embodiment, the container comprising the stem cells is a syringe. The syringe can be prepared so that its contents remain aseptic and ready for injection, e.g., by merely attaching a needle to the syringe. The kit can further comprise a second container, the second container comprising a supplemental composition for introducing into the site of damage together with the stem cells. Examples of supplemental compositions include, but are not limited to platelet-rich plasma (see U.S. Pat. No. 6,811,777), growth factors, and IRAP (interleukin-1 receptor antagonist protein. IRAP blocks IL-1 from binding to tissues and inhibits the damaging consequences of IL-1). In one embodiment, the second container is a chamber attached to the first container. For example, where the first container is a syringe, the second container can be attached to the syringe, its contents separated from the contents of the first container by a destructible barrier. Upon breach of the barrier, the contents of the second container enter into the first container and mix with the stem cells, for injection as a single composition.

Kits of the invention optionally further comprise instructions for use in accordance with one or more methods of the invention. The instructions can be provided in print form or via other media, including, for example, a computer readable disc, such as a digital video disc, portable drive, memory card, or compact disc.

### Therapeutic Use of Pluripotent Cells

The PC of the invention can be implanted into the site of a host in need of tissue repair, including connective tissue, other sites outside the central nervous system (CNS) or intravenously. Conditions for successful transplantation include: 1) viability of the implanted cells; 2) differentiation into appropriate phenotypic expression, such as into fibers that align along the long axis of the tendon; and 3) minimum amount of pathological reaction at the site of transplantation. Typically, the transplantation is by injection into the site of damage or intra-venous.

Therapeutic use of PC can be applied to ameliorate symptoms of connective tissue damage. Examples of connective tissue damage include, but are not limited to, ligament damage, osteochondrosis, tendonitis, navicular syndrome damage, arthritis, laminitis or cartilage damage.

Typically, the vertebrate subject is a mammalian or avian, and includes primates (including humans), equine, bovine, ovine, porcine, canine, feline, and other veterinary subjects. In one embodiment, the subject is a horse. Typically, the subject or recipient of transplanted PC of the invention is of the same species as the PC. The PC are MHC-negative and suitable for allogeneic transplant.

The tissue damage includes damage due to disease or injury. In a typical embodiment, the tissue is connective tissue. In one embodiment, the introducing is by injection into the site of damage. The injection can be performed under ultrasound guidance. In another embodiment, the introducing is by implanting the cells into an area that communicates with the site of damage such that the stem cells arrive at the site of damage by migration or via the circulatory system.

In one embodiment, one or more doses of the invention are introduced into the site of damage. A dose can comprise from about 0.5 to about 10 million stem cells, and in most cases, about 1 to 2 million cells. In a typical embodiment for an equine subject presenting with connective tissue damage, 1.2 million stem cells are introduced per dose. A given dose can be expected to diminish by 5-10% due to loss of cell viability during transport, such that an initial dose of 1.2 million cells may actually result in the administration of approximately 1 million live cells. Those skilled in the art understand that the dose can be increased or decreased to accommodate use with individual subjects, taking into account the subject's size and the nature of the disease or injury to be treated. Example 12 below describes typical doses for use with canine subjects. Felines and other smaller animals can be treated with fewer cells, while animals larger than equine subjects can be treated with larger doses.

The cells can be derived from equine fetal tissues, e.g., whole brain and spinal cord. Cells derived from other vertebrate species (e.g., canine, feline, etc.) are taken from tissue of the corresponding gestational age. The amount of cells used is typically constrained by volume, both in terms of a suitable volume for injection and constraints of the site into which the cells are to be injected. An implantation of 1,200,000 cells has been found sufficient to achieve suitable results, even where far fewer cells were needed. Any excess cells are cleared from the site by apoptosis and phagocytosis, and no evidence has been found of implanted cells that failed to either migrate to a site of disease or damage or be cleared.

Methods for transplanting various neural tissues into host brains are described in U.S. patent application Ser. No. 11/002,933, filed Dec. 2, 2004. Those skilled in the art will appreciate the ability to adapt transplantation methods described in the published patent application as well as the methods detailed herein for use with other sites of treatment.

The cellular suspension procedure permits grafting of PC to any predetermined site or intra-venous injection (in case of a diffuse wide-spread disease), is relatively non-traumatic, allows multiple grafting simultaneously in several different sites or the same site using the same cell suspension, and permits mixtures of cells having different characteristics. Typically, the graft consists of a substantially pure population of PC.

### Genetically Modified PC

Although one advantage of the PC of the invention is the ability to use them without pre-differentiation or genetic modification, these cells are amenable to genetic modification. In some embodiments, the present invention provides methods for genetically modifying PC for grafting into a target tissue site or for use in screening assays and the creation of animal models for the study of disease conditions.

In one embodiment, the cells are grafted into the site of damage to treat defective, diseased and/or injured cells. The methods of the invention also contemplate the use of grafting of transgenic PC in combination with other therapeutic procedures to treat disease or trauma. Thus, genetically modified PC of the invention may be co-grafted with other cells, both genetically modified and non-genetically modified cells, which exert beneficial effects on cells in the site to be treated. The genetically modified cells may thus serve to support the survival and function of the co-grafted, non-genetically modified cells. Moreover, the genetically modified cells of the invention may be co-administered with therapeutic agents useful in treating defects, trauma or diseases, such as growth factors, gangliosides, antibiotics, neurotransmitters, neuropeptides, toxins, neurite promoting molecules, and anti-metabolites and precursors of these molecules, such as the precursor of dopamine, L-dopa.

Vectors carrying functional gene inserts (transgenes) can be used to modify PC to produce molecules that are capable of directly or indirectly affecting cells to repair damage sustained by the cells from defects, disease or trauma. In one embodiment, for treating defects, disease or damage of cells, PC are modified by introduction of a retroviral vector containing a transgene or transgenes. The PC may also be used to introduce a transgene product or products that enhance the production of endogenous molecules that have ameliorative effects in vivo.

Those skilled in the art will appreciate a variety of vectors, both viral and non-viral, that can be used to introduce the transgene into the PC. Transgene delivery can be accomplished via well-known techniques, including direct DNA transfection, such as by electroporation, lipofection, calcium phosphate transfection, and DEAE-dextran. Viral delivery systems include, for example, retroviral vectors, lentiviral vectors, adenovirus and adeno-associated virus.

The nucleic acid of the transgene can be prepared by recombinant methods or synthesized using conventional techniques. The transgene may include one or more full-length genes or portions of genes.

Although those skilled in the art appreciate the advantages of using genetically modified PC, it is also appreciated that, in some embodiments, it is preferable to use a preparation of PC that is free of genetically modified cells. As described in U.S. patent application Ser. No. 11/755,224, filed May 30, 2007, and published Nov. 22, 2007, as US2007-0269412A1, transplanted PC of the invention, free of genetically modified cells or other cell types, are able to migrate to a site of damage or dysfunction and adopt a phenotype tailored to the needs of the damaged region. This has been observed in both an animal model of Parkinson's disease and an animal model of epilepsy. Epilepsy symptoms and damage have been treated in both rodent and canine subjects. Accordingly, the desired therapeutic effect can be achieved without any concerns that might be associated with use of transgenes and genetically modified cells.

### Administration and Dosage

The compositions are administered in any suitable manner, often with pharmaceutically acceptable carriers. Suitable methods of administering cells in the context of the present invention to a subject are available, and, although more than one route can be used to administer a particular cell composition, a particular route can often provide a more immediate and more effective reaction than another route.

The dose administered to a subject, in the context of the present invention, should be sufficient to effect a beneficial therapeutic response in the subject over time, or to inhibit disease progression. Thus, the composition is administered to a subject in an amount sufficient to alleviate, reduce, cure or at least partially arrest symptoms and/or complications from the disease or condition. An amount adequate to accomplish this is defined as a “therapeutically effective dose.”

Routes and frequency of administration of the therapeutic compositions disclosed herein, as well as dosage, will vary from individual to individual, and may be readily established using standard techniques. Typically, the pharmaceutical compositions are administered by injection. A single injection may suffice or, in some embodiments, between 1 and 5 doses may be administered, based on the judgment of the supervising veterinarian.

Alternate protocols may be appropriate for individual patients. Multiple sequential injections are possible because the stem cells of invention are hypo- or non immunogenic.

A suitable dose is an amount of a substance that, when administered as described above, is capable of promoting a therapeutic response, and is at least a 10-50% improvement relative to the untreated level. In general, an appropriate dosage and treatment regimen provides the material in an amount sufficient to provide therapeutic and/or prophylactic benefit. Such a response can be monitored by establishing an improved clinical outcome (e.g., more frequent remissions, complete or partial, or longer disease-free survival) in treated subjects as compared to non-treated ones. In a typical embodiment, improvement in the treated area is monitored monthly via ultrasound.

### Pharmaceutical Compositions

The invention provides pharmaceutical compositions comprising PC and, optionally a physiologically acceptable carrier. Pharmaceutical compositions within the scope of the present invention may also contain other compounds that may be biologically active or inactive. For example, one or more biological response modifiers may be present within the composition.

While any suitable carrier known to those of ordinary skill in the art may be employed in the pharmaceutical compositions of this invention, the type of carrier will vary depending on the mode of administration. Compositions of the present invention may be formulated for any appropriate manner of administration. Such compositions may also comprise buffers (e.g., neutral buffered saline or phosphate buffered saline), carbohydrates (e.g., glucose, mannose, sucrose or dextrans), mannitol, proteins, polypeptides or amino acids such as glycine, antioxidants, chelating agents such as EDTA or glutathione, adjuvants (e.g., aluminum hydroxide) and/or preservatives.

### EXAMPLES

The following examples are presented to illustrate the present invention and to assist one of ordinary skill in making and using the same. The examples are not intended in any way to otherwise limit the scope of the invention.

### Example 1

### Preparation of Progenitor Cells

The preparation of brain-derived pluripotent stem cells (BPC) is described in U.S. patent application Ser. No. 11/002,933, published Jun. 2, 2005 as publication number US2005-0118561. These same preparations, initially characterized as BPC, were later determined to have features and express markers associated with pluripotent cells. The BPC were derived from the telencephalon (T lines), mesencephalon (M lines) or whole fetal brain (B lines). Due to little or no differences observed between T, M and B lines, separate cultures for T and M lines proved unnecessary and cultures have henceforth been prepared using whole brain.

This example describes the preparation of cells from equine fetal brain. Subsequent studies have shown that the same preparation and culturing techniques are successful when used with fetal canine brain. Those skilled in the art will appreciate that the same techniques could likewise be adapted for use with other species, including, for example, felines.

Tissue was obtained from equine fetal tissue. Tissue samples were dissected from skin, cartilage, heart, liver, pancreas, lung, spinal cord and brain. The tissue was prepared and cultured as described previously for human fetal brain tissue. Of these tissues, the cells derived from liver, spinal cord, heart and brain survived best. After 60 days in culture, for example, liver-derived cells formed small, medium-sized, or large, irregularly-shaped floating clusters and exhibited little or no attachment to the culture surface. Skin-derived cells showed strong attachment and no floating cells at this point in time. The skin-derived cells grew initially and then died after 2 months, while the liver-, spinal cord- and brain-derived cells continued to grow indefinitely. Brain-derived cells, by 37 days in culture, showed some attachment and formed irregularly-shaped floating clusters amongst a single cell suspension. At 37 days in culture, spinal cord-derived cells showed strong attachment of spindle-shaped cells. By 3 months in culture, the brain-derived cells had become homogeneous, showing uniform expression of markers, and appeared as large, floating spherical clusters, much like embryoid bodies.

### Example 2

### Characterization of Source Tissue

Stem cells were obtained from a horse fetus. The fetal tissue was dissected under sterile conditions in Hanks Balanced Salt Solution (HBSS) supplemented with Gentamycin and Fungizone. After multiple washes (at least ten times in anti-microbial and anti-fungal HBSS), the tissue was minced with microscissors under a dissecting microscope in a laminar flow hood (Thermo Scientific, Fisher) and then triturated with sterile fire-polished Pasteur pipettes until a single cell suspension was obtained. Cell counts were performed using a hemocytometer. Cells were cultured in an incubator (Thermo Scientific, Fisher), approximately 10,000,000 cells per flask, for one week to confirm that there was no apparent contamination, as determined by examination under a light microscope. Samples of the cell culture were sent to an outside laboratory for karyotyping, and for genetic, microbial and viral screening. If results were negative, cell cultures remained in the manufacturing process.

### Stem Cell Culture and Immunostaining

Once sterility of cell culture was confirmed, cells were explanted into culture flasks at a final concentration of 400,000 cells/flask in the above culture medium. Once a week, cells were centrifuged, supernatant was discarded except 2 ml, in which cells were resuspended, transferred to the culture flask and 4 ml of fresh culture medium were added. Four days later, 4 mL of fresh culture medium was added to each flask. This cycle was repeated weekly. Cells were passaged 4-5 times before immunohistochemical testing for homogeneity and pluripotency.

To assess homogeneity and pluripotency, a 0.17 mL sample of cells were taken and centrifuged using the Centra CL2 Cytospin (Thermo Fisher IEC, Waltham, Mass.) to obtain a monolayer of cells on a microscope slide. Slides were fixed with 4% formaldehyde for 20 minutes, and permeabilized for one hour with 0.1% Triton-X. Cells were incubated with primary antibodies for anti-rabbit Oct-4 (1:200, Millipore AB3209), anti-mouse Major Histocompatibility Complex (MHC, 1:50, Invitrogen MM3415) anti-rabbit Nestin (1:250, Abcam AB5968), anti-rabbit Telomerase (1:200, Calbiochem 582005), and anti-mouse Ki-67 (1:50, Invitrogen 18-0192Z) overnight at room temperature. Anti-mouse (1:100, Vector BA-2001) or anti-rabbit secondary antibody (1:100, Vector BA-1000) was applied for two hours at room temperature. This was followed by Avidin-Biotin Kit (Vector PK-6100) for 30 minutes, and DAB complex (Dako K3468) for one minute.

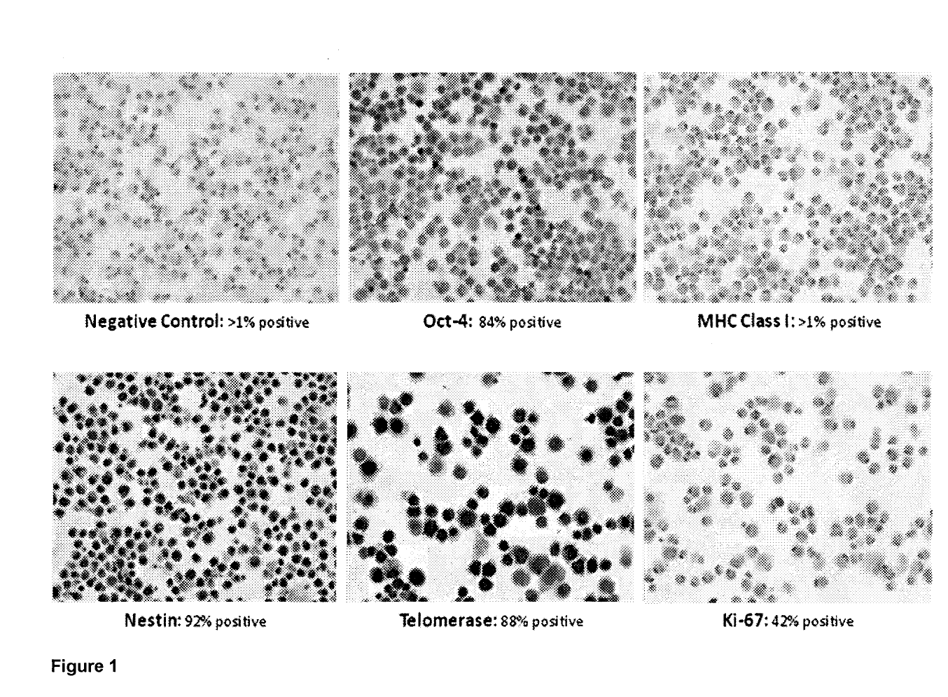

Once a cell culture expressed markers for pluripotency (Oct-4, TRA-1-60, TRA-1-81, SSEA-4, Nestin, Telomerase) and did not express any Major Histocompatibility Complex (MHC), they were determined to be suitable for transplant. (See FIG. 1 of Example 4, below).

To prepare cells for transplant, cells were counted using a hemocytometer, and one million cells were condensed into 2 mL culture media. Cells were packaged in a 2 mL cryovial and transported on ice to transplant site.

### Example 3

### Dosage & Transportation of the Cells

Stem cells are counted and repackaged in an optimal dose of 1.2 million cells/2 mL of proprietary culture medium for transportation. The amount of cells has been validated as optimal in our pilot clinical study in soft tissue injuries. Vials with stem cells are packaged with ice packs in a STYROFOAM™ container and shipped to the veterinarian for delivery within 24 hours.

The optimal dose for most injuries is 1.2 million cells, and will be referred to by that number throughout this application. The dose of 1.2 million cells is designed to allow for retention of about 1 million cells after some cells are lost in transport. Viability tests have shown that about 85% of the cells remain viable at 48 hours after coast-to-coast transport via overnight delivery in a STYROFOAM™ cooler. However, the final dose is always to be determined by the nature of the injury and the veterinarian.

The equine brain-derived PC cultured in the medium of the invention have been shown to have the characteristics of stem cells. By two months in culture, these cells are about 80% positive for telomerase, an indicator that a cell will divide repeatedly without aging. Only about 10% are immunopositive at this point for p53, a marker indicating the initiation of cell death. About 60% are immunopositive for Oct-4, a protein responsible for the self-renewal of stem cells. The cells show no positive staining for MHC Class I, a marker for the ability to elicit an immune response.

By 3 months in culture, the fetal equine whole brain-derived cells are about 99% immunopositive for telomerase, and about 85% immunopositive for Oct-4. They remain immunonegative for MHC Class I.

### Example 4

### Transplantation of PC into Injured Horse Hind Limb

This example demonstrates that PC prepared in accordance with the invention, taken from 39 day old equine fetus (22 mm CRL), can be successfully grafted into damaged equine superficial digital flexor tendon and effect histological recovery. Initial treatments were provided to a retired horse with experimental deep tendon injury induced by a 20 mm diameter vertical hole placed in the tendon of all four legs in double blind controlled fashion. In the double-blind study, the tendon of each leg was injected with either 5 million skin-derived cells, 3 million brain-derived cells, 10 million brain-derived cells or vehicle control. Within 5 weeks, near normal linear pattern of the tendon was restored as documented via ultrasound. Ultrasound study at 1 month showed that the tendons in two legs treated with brain-derived cells regained near normal structure in place at the site of prior injury. Initially, comparisons were made between skin-derived cells and brain-derived cells. Due to superior results observed with brain-derived cells, studies were limited to use of brain-derived cells for the first treatments.

By 4 months after treatment, the first horse, who was more than 20 years of age, showed no rejection of the transplanted cells and anatomically correct healing with this allogeneic transplant. A horse that previously could not walk without a heavy dose of painkillers had reacquired normal gait without medications.

### Example 5

### Injection of PC into Equine Tendon and Ligament for Treatment of Injury

This example demonstrates that PC prepared in accordance with the invention can be successfully grafted into damaged equine hind limb and effect histological recovery. 63 performance horses were referred for tendon and ligament injury. Treatment of the affected tendons and ligaments with PC of the invention shows early promise of quality healing.

The present study demonstrates treatment with PC as an effective alternative regenerative therapy for tendon and ligament injuries. Embryonic derived stem cells are pluripotent and non-immunogenic, which gives them the capability to generate almost any type of cells without the danger of immune mediated rejection. Ultrasonography suggests that the tendons or ligaments heal with a matrix more like the original tissue and less like scar tissue. Horses with tendonitis and torn ligaments that are treated with embryonic derived stem cells are able to resume training sooner with a better quality of healing.

### Materials and Methods

### Case Details and Diagnostic Findings

63 horses, ranging from 3-20 years old, one week to 36 months after injury, were presented for investigation of suspected ligament or tendon injuries. Owners had mostly noted the horses had incurred an injury manifested by swelling, pain, lameness or combination thereof. On presentation the horses were examined clinically, and by ultrasonography to determine extent of the injury.

Of the 63 subjects, 42 were male and 21 were female. The breeds included 45 quarter horses, 16 thoroughbreds and 2 “other”. The types of injuries included 23 acute tendon, 8 chronic tendon, 16 acute ligament, and 16 chronic ligament.

### Ultrasonography

Ultrasonographic imaging of the suspected injury site was performed using a 10 MHz linear transducer. The lesions were then described as follows: 21 tendon tears, most showing diffuse loss of fiber pattern in Zones 2 or 3, with initial examination occurring from one week to 3 years post-injury; and 11 ligament lesions, with initial examination occurring anywhere from three days to 3 years post-injury.

### Stem Cell Procurement

Equine fetuses were obtained at 39-42 days gestation by flushing. Sample was then washed multiple times in HBSS supplemented with Gentamycin (MP Biomedicals 1676045, Solon, Ohio) and Fungizone (Omega Scientific FG-70, Tarzana, Calif.). The fetal tissue (specifically brain, spinal cord, liver and heart) was dissected under sterile conditions in Hanks Balanced Salt Solution (HBSS, Invitrogen 14025, Carlsbad Calif.) supplemented with Gentamycin and Fungizone. After multiple washes (at least ten times in anti-microbial and anti-fungal HBSS), each organ was separately minced with microscissors under a dissecting microscope (Olympus SZ61, Center Valley, Pa.) in a laminar flow hood (1846, Thermo Scientific, Waltham, Mass.) and then triturated with sterile fire-polished Pasteur pipettes until a single cell suspension was obtained.

Cell counts were performed using a hemocytometer, and viability was tested using Trypan Blue exclusion and documented in laboratory notebooks. Each tissue sample produced approximately 40 million cells. These cells were explanted into culture flasks at a final concentration of ˜10 million cells/flask, in culture medium containing the following components:

### Formulation of Culture Media

| Component: | Concentration: | |
|---|---|---|
| EMEM (Eagle's Essential Media, Lonza RR116254, | 100 | mL |
| Walkersville MD) | | |
| B27 (Invitrogen 17504, Carlsbad, CA) | 2.0% | |
| Calcium Chloride (Fisher Scientific, Pittsburgh, PA) | 0.05 | mM |
| Epidermal Growth Factor (Peprotech 100-15, Rocky | 40 | ng/mL |
| Hill, NJ) | | |
| Basic Fibroblast Growth Factor (Peprotech 100-18B, | 40 | ng/mL |
| Rocky Hill, NJ) | | |
| Transforming Growth Factor Alpha (Peprotech 100-16A, | 40 | ng/mL |
| Rocky Hill, NJ) | | |
| Leukemia Inhibitory Factor (Millipore LIF1010, | 50 | ng/mL |
| Temecula, CA) | | |
| Glutamax ™ (Invitrogen, Carlsbad, CA) | 0.1 | mM |

| | |
|---|---|
| N2 Supplement (Invitrogen 17502, Carlsbad, CA) | 1.0% |

Cells were cultured in an incubator (Thermo Scientific, Fisher) for one week to determine that there was no contamination, as defined by examination under a light microscope. Samples of the cell culture were sent to an outside laboratory for microbial and viral screening (Bionique Testing Laboratories, Saranac Lake, Calif.). If results are negative, cell cultures remain in the manufacturing process.

### Stem Cell Culture and Immunostaining

Once sterility of cell culture was confirmed, cells were explanted into culture flasks at a final concentration of 400,000 cells/flask in the above culture medium. Once a week, cells were centrifuged, supernatant was discarded except 2 ml, in which cells were resuspended, transferred to the culture flask and 4 ml of fresh culture medium were added. Four days later, 4 mL of fresh culture medium was added to each flask. This cycle was repeated weekly. Cells were passaged 4-5 times before immunohistochemical testing for homogeneity and pluripotency.

To assess homogeneity and pluripotency, a 1.7 mL sample of cells were taken and centrifuged using the Centra CL2 Cytospin (Thermo Fisher IEC, Waltham, Mass.) to obtain a monolayer of cells on a microscope slide. Slides were fixed with 4% formaldehyde for 20 minutes, and permeabilized for one hour with 0.1% Triton-X. Cells were incubated with primary antibodies for anti-rabbit Oct-4 (1:200, Millipore AB3209), anti-mouse Major Histocompatibility Complex (MHC, 1:50, Invitrogen MM3415) anti-rabbit Nestin (1:250, Abcam AB5968), anti-rabbit Telomerase (1:200, Calbiochem 582005), and anti-mouse Ki-67 (1:50, Invitrogen 18-0192Z) overnight at room temperature. Anti-mouse (1:100, Vector BA-2001) or anti-rabbit secondary antibody (1:100, Vector BA-1000) was applied for two hours at room temperature. This was followed by Avidin-Biotin Kit (Vector PK-6100) for 30 minutes, and DAB complex (Dako K3468) for one minute.

Once a cell culture expressed markers for pluripotency (Oct-4, Nestin, Telomerase) and did not express any Major Histocompatibility Complex (MHC), they were determined to be suitable for transplant. (See FIG. 1).

### Cell Preparation for Transplant

To prepare cells for transplant, cells were counted using a hemocytometer, and one million cells were condensed into 2 mL culture media. Cells were packaged in a 2 mL cryovial and transported on ice to transplant site.

### Treatment

Management options were discussed with the owners of each case. These options consisted of athletic rest with no proactive therapy, or injecting the lesion with embryonic stem cells combined with rest and rehabilitation. When owners chose the latter, a stem cell injection was prepared and transported to site. A total of 77 treatments were administered.

Sedation was provided by a combination of detomidine HCl (6 mg/kg bwt) and butorphanol tartrate (10 mg/kg bwt) administered i.v. A 20 mg/kg bwt dose of Dexamethasone Sodium Phosphate was additionally administered intravenously.

The affected area was finely clipped and surgically prepped using a combination of Betadine scrub and 70% Isopropyl alcohol diluted 1:10 with 4% chlorhexidine gluconate.

PC were aseptically transferred from transport vial to a 3 cc luer lock syringe using a 22 gauge×1.5″ needle.

The affected limb was held in a non-weight bearing stance and the lesion was identified by ultrasonographic imaging using a sterile wrapped 10 MHz linear transducer. A 22 gauge×1.5″ needle was placed thru the palmar aspect of the skin directly into the lesion. One million PC suspended in 2 cc were injected directly into the lesion as identified by ultrasonographic assessment.

Post-injection ultrasonography was performed to ensure accuracy of injection, and a sterile dry Robert-Jones bandage was applied from distal to the carpus to the coronary band.

### Post Operative Management

No antibiotic therapy or pain management drugs were administered. Treated horses remained hospitalized for 3 days following the procedure. The sterile bandage was removed 2 days after the procedure. Treated subjects were discharged from the hospital with aftercare instructions consisting of strict stall rest with 10-15 minutes of hand walking daily until the 30 day re check examination. No bandages or medications were indicated once released from the hospital.

### Results

### Post-Op Follow-Up

Two days after procedure was performed the sterile bandage was removed, and horse was evaluated for swelling and/or inflammation. All 63 treated cases have shown no signs of rejection, inflammation, or painful swelling 2 days after injection, and all treated horses have been released from the hospital with no indications for further medication.

### 30 day Follow-Up

An ultrasonic examination was performed 30 days post procedure for horses treated with stem cell injection. To date, all 30-day ultrasonic follow-ups have shown no signs of rejection or inflammation. By this time point, most horses have shown overall improvement in lesion size and fiber alignment. Owners of these horses were instructed to increase exercise level by track walking and trotting for 20 minutes daily. Some of the horses showed no significant improvement after thirty days, and most of these cases were treated with a second dose of stem cells, which again showed no signs of rejection or inflammation post-operatively.

### Further Follow-Up

Calculations were done for all four types of injured tissue: acute and chronic, tendon and ligament. The following measures were evaluated: lameness, echogenicity, fiber alignment, exercise levels and pain. The treatments were found to be safe, as there were no signs of rejection, tumor formation, infectious complications or other significant complications.

Anatomic restoration and regeneration of injured tendon and ligament tissue was confirmed by diagnostic ultrasound, which showed improvements in echogenicity and fiber alignment. Improvements were also observed in exercise levels, with lameness and pain levels both reduced. Also observed was a lack of scar tissue, which would normally inhibit performance. Most subjects were able to return to full training within 120 days.

Fiber alignment, as shown via ultrasound, was rated on a 0-4 point scale. Measures were taken at days 0, 30, 60, 90 and 120 after treatment. Average scores were as follows:

| n = number of | | | | | |
|---|---|---|---|---|---|
| ligaments/tendons | Day 0 | Day 30 | Day 60 | Day 90 | Day 120 |
| Ligaments | 2.6 | 2.23 | 1.43 | 1.8 | 0.33 |
| | n = 20 | n = 13 | n = 14 | n = 10 | n = 3 |
| Tendons | 2.98 | 2.31 | 1.44 | 1.44 | 1.0 |
| | n = 25 | n = 16 | n = 16 | n = 9 | n = 7 |

Fiber alignment scores, separately viewed as acute and chronic tendon injuries, were as follows:

| n = number | | | | | |
|---|---|---|---|---|---|
| of tendons | Day 0 | Day 30 | Day 60 | Day 90 | Day 120 |
| Acute | 2.89 | 2.15 | 1.08 | 0.5 | 0.4 |
| | n = 19 | n = 13 | n = 12 | n = 4 | n = 5 |
| Chronic | 3.25 | 3.0 | 2.5 | 2.2 | 2.5 |
| | n = 6 | n = 3 | n = 4 | n = 5 | n = 2 |

Echogenicity was also rated on a 0-4 point scale, with measures taken at days 0, 30, 60, 90 and 120 after treatment. Average scores were as follows:

| n = number of | | | | | |
|---|---|---|---|---|---|
| ligaments/tendons | Day 0 | Day 30 | Day 60 | Day 90 | Day 120 |
| Ligaments | 2.45 | 2.08 | 1.93 | 2.1 | 1.5 |
| | n = 20 | n = 13 | n = 15 | n = 10 | n = 4 |
| Tendons | 2.94 | 2.29 | 1.44 | 1.67 | 1.14 |
| | n = 25 | n = 17 | n = 16 | n = 9 | n = 7 |

Echogenicity scores, separately viewed as acute and chronic tendon injuries, were as follows:

| n = number | | | | | |
|---|---|---|---|---|---|
| of tendons | Day 0 | Day 30 | Day 60 | Day 90 | Day 120 |
| Acute | 2.84 | 2.15 | 1.08 | 1.0 | 1.0 |
| | n = 19 | n = 13 | n = 12 | n = 4 | n = 5 |
| Chronic | 3.25 | 2.75 | 2.5 | 2.2 | 1.5 |
| | n = 6 | n = 4 | n = 4 | n = 5 | n = 2 |

Lameness was rated on a 0-5 point scale, with measures taken at days 0, 30, 60, 90 and 120 after treatment. Average scores were as follows:

| n = number of | | | | | |
|---|---|---|---|---|---|
| ligaments/tendons | Day 0 | Day 30 | Day 60 | Day 90 | Day 120 |
| Ligaments | 1.62 | 1.05 | 0.79 | 0.46 | 0.5 |
| | n = 26 | n = 19 | n = 19 | n = 13 | n = 6 |
| Tendons | 1.85 | 2.27 | 1.56 | 1.45 | 1.24 |
| | n = 20 | n = 13 | n = 9 | n = 7 | n = 7 |

Lameness, separated by acute versus chronic ligament injuries, was rated as follows:

| n = number | | | | | |
|---|---|---|---|---|---|
| of ligaments | Day 0 | Day 30 | Day 60 | Day 90 | Day 120 |
| Acute | 2.0 | 1.17 | 0.73 | 0.25 | 0.33 |
| | n = 26 | n = 19 | n = 19 | n = 13 | n = 6 |
| Chronic | 1.17 | 0.86 | 0.88 | 0.8 | 0.67 |
| | n = 20 | n = 13 | n = 9 | n = 7 | n = 7 |

Exercise level was determined to be at one of 7 levels, based on an 11-point scale, with measures taken at days 0, 30, 60, 90 and 120 after treatment. Average scores were as follows:

| n = number of | | | | | |
|---|---|---|---|---|---|
| ligaments/tendons | Day 0 | Day 30 | Day 60 | Day 90 | Day 120 |
| Ligaments | 1.04 | 2.18 | 3.84 | 5.62 | 5.67 |
| | n = 24 | n = 17 | n = 19 | n = 13 | n = 6 |
| Tendons | 1.52 | 1.5 | 2.88 | 4.2 | 5.44 |
| | n = 21 | n = 18 | n = 16 | n = 10 | n = 9 |

Average scores of exercise levels, separated by acute versus chronic tendon injuries, were as follows:

| n = number | | | | | |
|---|---|---|---|---|---|
| of tendons | Day 0 | Day 30 | Day 60 | Day 90 | Day 120 |
| Acute | 1.07 | 1.29 | 3.0 | 4.5 | 6.83 |
| | n = 15 | n = 14 | n = 13 | n = 6 | n = 6 |
| Chronic | 1.53 | 2.4 | 2.09 | 2.89 | 4.43 |
| | n = 6 | n = 4 | n = 3 | n = 4 | n = 3 |

Exercise level scores, separated by acute versus chronic ligament injuries, were as follows:

| n = number | | | | | |
|---|---|---|---|---|---|
| of ligaments | Day 0 | Day 30 | Day 60 | Day 90 | Day 120 |
| Acute | 1.0 | 2.08 | 5.18 | 7.75 | 6.67 |
| Chronic | 1.08 | 2.4 | 2.0 | 2.2 | 4.67 |

Pain level was rated yes (1) or no (0), with measures taken at days 0, 30, 60, 90 and 120 after treatment. Average scores were as follows:

| n = number of | | | | | |
|---|---|---|---|---|---|
| ligaments/tendons | Day 0 | Day 30 | Day 60 | Day 90 | Day 120 |
| Ligaments | 0.48 | 0.41 | 0.15 | 0.08 | n = 0 |
| | n = 12 | n = 10 | n = 10 | n = 5 | |
| Tendons | 0.52 | 0.29 | 0.11 | 0.11 | 0.1 |
| | n = 31 | n = 24 | n = 18 | n = 10 | n = 10 |

Average scores of pain levels, separated by acute versus chronic tendon injuries, were as follows:

| n = number | | | | | |
|---|---|---|---|---|---|
| of tendons | Day 0 | Day 30 | Day 60 | Day 90 | Day 120 |
| Acute | 0.54 | 0.22 | 0 | 0 | 0 |
| | n = 24 | n = 18 | n = 13 | n = 5 | n = 6 |
| Chronic | 0.43 | 0.5 | 0.4 | 0.4 | 0.25 |
| | n = 7 | n = 6 | n = 5 | n = 5 | n = 4 |

Swelling level results, also scored on a yes (1) or no (0) basis, separated by acute versus chronic ligament injuries, were as follows:

| n = number | | | | | |
|---|---|---|---|---|---|
| of ligaments | Day 0 | Day 30 | Day 60 | Day 90 | Day 120 |
| Acute | 0.73 | 0.83 | 0.2 | 0 | 0 |
| | n = 15 | n = 12 | n = 10 | n = 7 | n = 3 |
| Chronic | 0.25 | 0.2 | 0.1 | 0 | 0 |
| | n = 12 | n = 10 | n = 10 | n = 5 | n = 4 |

### CONCLUSION

Results of this study confirm the ability of PC of the invention to restore the near normal structure of an injured tendon or ligament, with no indications of inflammation or rejection after treatment. The safety of this treatment has been demonstrated by no rejection, no tumor formation, no infectious complications and no other significant complications. The diagnostic ultrasound results indicate improved and sustained regenerative effects of PC treatment for both acute and chronic tendon injury and for both acute and chronic ligament injury. The results also confirm a lack of scar tissue inhibiting performance, and a return to full training within 120 days for most subjects followed to this time point post-transplant.

### Example 6

### Stem Cell Transplantation Kit for Regenerative Veterinary Therapy

A kit has been prepared for regenerative veterinary therapy using stem cells of the invention. The kit contains one or more doses (depending on request of veterinarian), each dose typically provided in a vial, and each containing 2.0 mL of the culture medium of the invention and approximately 1,200,000 equine-derived stem cells. The kits are prepared for transport after feeding and counting the cells using conventional protocols. Using sterile technique in a laminar flow hood, the desired number of cells is placed into a cryovial and topped off with fresh culture medium to a total volume of 2 ml. The vial is placed in a STYROFOAM™ (Dow Chemical Company) cooler containing 2-3 ice packs. Vial or syringe is arranged so as to remain in an upright position and without direct contact with ice packs. A specification sheet and instructions for use are included, the cooler is sealed and transported to the site of use via overnight delivery. Cells remain viable for up to 48 hours in this condition. Tests have shown 85% viability at 48 hours for cells that have been shipped coast-to-coast across the United States. The suggested dose of 1,200,000 cells takes into account the small loss of viability observed after delivery.

The cells have undergone a thorough immunohistochemical and microbial screening. They do not express MHC I or II, nor do they express p53. They are immunopositive for telomerase, Oct-4, TRA-1-60, TRA-1-81, SSEA-4 and Nanog. This characterization confirms the cells' potential to form various restorative cell types while remaining immunologically inactive, thus avoiding inflammation or rejection from the host post-transplant.

The kits optionally includes a set of detailed instructions. First, the sterile syringe is removed from the cooler immediately before use. It is recommended that the cells be used immediately upon delivery, as they lose viability over time after removal from the cultivation environment. The solution should be cold, but not frozen at the time of injection. The injection site is cleaned, e.g., with betadine and antibiotic, using sterile surgical technique. Using the sterile 2 cc syringe provided and a 22 gauge needle, one complete syringe of cells is transplanted into each damaged area using ultrasound-guided injection, taking care to fill the entire affected area. Sterile technique is to be maintained during transplant. Additional vials are used in the same manner for other areas of damage or other animals requiring treatment. Post-operatively, the transplanted area should be monitored weekly using ultrasound to monitor improvement.

### Example 7

### Cryopreservation of PC

Media ingredients were varied and manipulated to determine the optimal conditions for cryopreservation of PC. B27, in addition to DMSO, appears to provide a significant protective effect contributing to the exceptionally high viability observed in thawed PC.

After the number of stem cells in the incubator reaches 40,000,000, all but 10,000,000 cultured cells were harvested and frozen in liquid Nitrogen. Cryo medium contains the expansion culture medium with 10% DMSO, 4% of B-27 supplement, and 0.5% of MEM non-essential amino acids solution (Gibco, N.Y.).

The step-by-step protocol for freezing cells is as follows:
1. Turn on laminar flow hood at least ten minutes prior to use.
2. Before performing any work in the hood, apply gloves and face mask, and spray hands and arms with 70% alcohol. Complete all work in hood under sterile conditions.
3. Prepare cryopreservation media by adding the following ingredients together into a 50 mL Falcon™ tube:
   72% fresh culture media (see Example 5 above)
   20% DMSO, sterile filtered (using a 0.25 μm filter) prior to use
   7% B27 supplement
   1% Non-essential amino acids.
4. After flask has been separated for freezing, feed flask 100% with normal culture media (NOT cryopreservation media).
5. Label cryovials with flask number, cell type, and date frozen.
6. Count cells in flask.
7. Once cells have been counted, move cell suspension back to 15 mL centrifuge tube, and centrifuge again for 5 minutes at 1000 rpm.
8. Remove tube from centrifuge. Using a sterile fire polished pipette, remove and discard all but 1 mL culture media from tube.
9. Resuspend cell pellet into 1 mL of remaining culture media.
10. Add 1 mL cryopreservation media to cell suspension.
11. Transfer 2 mL (total 1 mL cell suspension+1 mL cryopreservation media) from centrifuge tube to a sterile 1.8 mL cryovial.
12. Close cryovial tightly.
13. Repeat with all flasks to be frozen.
14. Transfer cryovials for −20° C. vial for 30 minutes. Set timer to ensure that cryovials are kept in freezer no longer than 30 minutes.
15. Transfer cryovials to −70° C. freezer overnight.
16. The next day, move cryovials to liquid nitrogen cryotank, attempting to move vials as quickly as possible to prevent thawing while moving. Do not allow vials to sit in −70° C. freezer longer than 24 hours.

For thawing, both the culture medium and the flask are pre-warmed to 37° C. in a water bath at 37° C. Using this cryopreservation method, over 95% viability is consistently observed in the PC upon thawing (using dye exclusion cell counts). Typically, the cells appear shrunken and of abnormal morphology for the first 5-7 days after thawing. Despite this appearance, the cells are able to exclude trypan blue dye. After about one week, the cells recover to their pre-freezing state, exhibiting typical morphology, growth and doubling times.

### Example 8

### Pluripotent Cells Derived from Heart and Liver

Cells prepared as described above in Example 1 and derived from heart and liver tissue initially appeared more heterogeneous and more difficult to establish in long-term culture. These cultures exhibited a longer period of time with fibroblast contamination. This was true for cultures established from heart and liver tissue of human, equine and canine origin, but especially for human tissue, which took 6-7 months to become homogeneous. At 2-3 months, the cultures showed both embryoid bodies and large, irregularly shaped clusters. Eventually, however, the cultures became predominantly floating embryoid bodies. These cultures show the same morphology and same pattern of marker expression observed for brain-derived cultures. They are negative for MHC, positive for telomerase, Oct-4, TRA-1-60, TRA-1-81, SSEA-4 and Nanog, and even about 30% of the cells are positive for nestin. Accordingly, these studies show that heart, liver, spinal cord and brain are all suitable source tissues for pluripotent stem cells of the invention.

### Example 9

### Treatment of Severe Arthritis in Canine Subjects Using Pluripotent Stem Cells

Osteoarthritis (OA) is the most common cause of chronic pain in dogs, with more than 20%, or 10 to 12 million dogs suffering from this condition at any time. OA is characterized by degeneration of the articular cartilage, with a loss of matrix, fibrillation, and formation of fissures. This can result in complete loss of the cartilage surface. In OA, there exists an overproduction of destructive and proinflammatory mediators relative to the inhibitors, resulting in a balance in favor of catabolism rather than anabolism. This is turn leads to the progressive destruction of articular cartilage.

The current cornerstone of care for OA in dogs is nonsteroidal anti-inflammatory (NSAIDs) drugs. In clinical practice, and as documented in the literature (Budsberg et al., 1999; Johnson et al., 1997) these pharmacological agents do not provide complete pain relief to the afflicted animals and remain lame. It is not unusual for owners of dogs with OA to consider euthanasia due to the animal's pain and functional disability.

Cells prepared as described above were administered intravenously to treat two 17- and 15 year old canine subjects afflicted with severe arthritis. The first subject had been treated with large doses of prednisone and painkillers and was immobile. The subject was given an intravenous administration of 1 million stem cells in lactate ringer's solution (this amount was diluted from 3 million cells to avoid anaphylactic shock). At one week after treatment, the dog needed help with one of its limbs, but otherwise could ambulate. One month after treatment, this dog is running around without difficulty using all 4 limbs. In addition to losing its arthritic gait, the dog has a shinier coat and now responds to commands, all significant improvements over its condition prior to treatment.

The second subject is a deaf, arthritic and partially blind 15-year-old canine exhibiting difficulty with spatial orientation. The dog was pre-treated with 6 mg dexamethasone and 14 mg diphenhydramine 35 min. prior to intravenous administration of 2 million stem cells in 60 ml of lactate ringer's solution. The subject has shown no side effects during the first 10 days after treatment, no longer bumps into objects in its environment, and now responds to commands.

### Example 10

### Distal End Perfusion for Intravenous Treatment Using Pluripotent Stem Cells

For injuries that involve a large, diffuse area, direct injection into the site of injury may not be optimal. Distal end perfusion offers a means of intravenous treatment with pluripotent cells that can deliver the PC to a larger area and/or treat distributed sites while still directing treatment to one limb. Administering the cells by distal end perfusion avoids potential confounding factors associated with whole-body systemic treatment.

Cells prepared as described above were administered intravenously to treat two legs of a horse suffering from diffuse degeneration of the suspensory ligament. A tourniquet was applied well-above the ligament with a large area of diffuse degeneration on the horse's right hind leg. While the leg was compressed with the tourniquet, 2 million stem cells in 60 ml lactate ringer's solution was delivered intravenously over a period of 8-10 minutes. The left hind limb was treated in the same fashion. The subject was pre-treated with 2 shots of dexamethasone 20 min. before stem cell injection. No side effects have been observed during the first 6 days after treatment. Follow up at 120 days post-treatment shows the horse doing well.

### Example 11

### Pluripotent Stem Cells Repair Equine Tendinitis/Suspensory Desmitis

This example shows that pluripotent stem cells of the invention improve tendinitis repair better than previous biologics, pharmaceuticals, or adult marrow and fat-derived stem cells.

Medical treatment with BAPTEN® (β-aminoproprionitrile fumerate), a therapy that had been approved by the FDA, has been described in Reef et al., 1997, AAEP Proceedings 43:301-305. This therapeutic resulted in a rapid reduction in tendon size and lameness levels, and ultrasonography showed improvement in lesion size and echogenicity compared to controls who were treated with an exercise program alone. Re-bow after racing occurred in 47% of cases, and 26% showed sustained racing. The treatment required a stringent protocol that included a rigid exercise program to be maintained for about a year. The product was discontinued for lack of interest.

The harvesting of bone marrow mesenchymal stem cells (MSC) for tendon repair has been described in Schnabel et al., 2009, J. Orthop. Res. 27(10):1392. No improvement was observed in the horses treated with MSC in ultrasonographic scores, in content of DNA, collagen or proteoglycans, in biomechanical characteristics, or in histologic scores.

In another study by Nixon et al. (2008, Am. J. Vet. Res. 69(7):928-937), an adipose vasculo-stromal cell fraction was harvested and used for tendon repair in horses with collagenase-induced tendinitis. Measures of collagen types I and II, and decorin gene expression, as well as ultrasonography, showed no improvement when comparing experimental animals to PBS-treated controls. The only improvement observed was in several histologic measures of tendon architecture.

The present study examined treatment of tendinitis with fetal brain-derived stem cells. Each of 8 equine subjects were assigned to one of two groups: 4 were injected with embryonic stem cells derived from fetal equine brain in accordance with the invention, and 4 were injected with control medium (saline). All subjects received an injection of collagenase in accordance with a collagenase gel tendinitis model (G. H. Spurlock et al., 1989, “Ultrasonographic, Gross, and Histologic Evaluation of A Tendinitis Disease Model In The Horse”, Veterinary Radiology & Ultrasound, 30 (4):184-188). All injections were performed under ultrasound guidance. Serial ultrasonography was performed at 0, 2, 4, 6 and 8 weeks. Horses were euthanized at 8 weeks and the superficial digital flexor tendon (SDFT) harvested for MRI, histology and immunohistochemistry, gene expression and DNA and collagen quantification. Analysis was performed by persons blind to whether the material was from an experimental or control subject.

Consistent, similar results were observed across all subjects within a group. The subjects receiving the stem cells showed improved ultrasound and MRI parameters assessing tendon healing. The experimental group also showed improved gross histological appearance, improved histological characterization of tendon fiber architecture, including crimp and uniformity, and with no significant increase in inflammatory cell infiltrate.

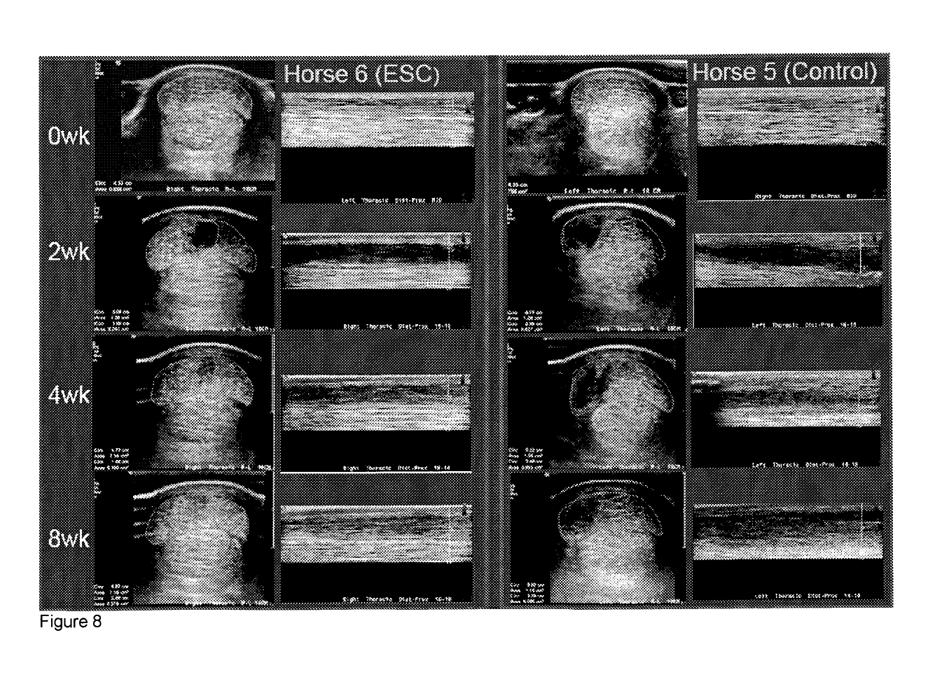

FIG. 8 is a series of sonograms from the ultrasound assessment of a representative subject from the experimental (Horse 6) and control (Horse 5) groups, taken at 0, 2, 4 and 8 weeks following collagenase injection. The tendon is shown in both cross-section (left columns) and longitudinal views (right columns). At 2 weeks, the collagenase-induced lesion is visible in both the control and experimental subjects. By 4 weeks, significant improvement is seen in the experimental subjects, but not in the controls. At 8 weeks, not only is the wound healed, but it exhibits remarkable fiber alignment, in contrast with the disorganized scar tissue observed in the control subject.

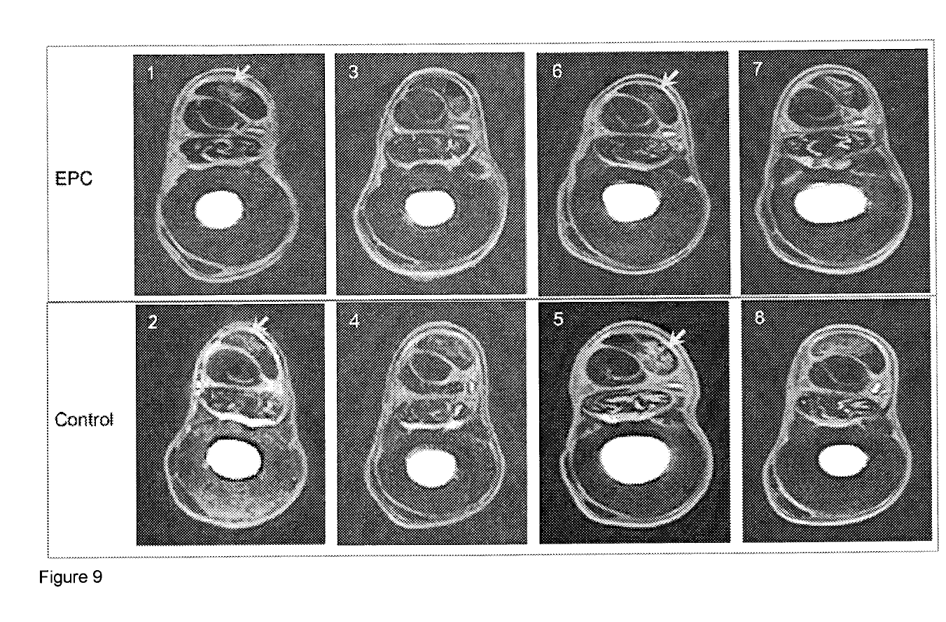

FIG. 9 is a series of cross-sectional images of the affected tendon viewed via MRI, which is more sensitive to scar tissue. The top row shows images from each of the experimental subjects taken 8 weeks following collagenase injection. Shown in the lower row are images from each of the control subjects, taken at the same time point. The arrows point to the area of disorganized (scar) tissue, which is significantly more present in control subjects.

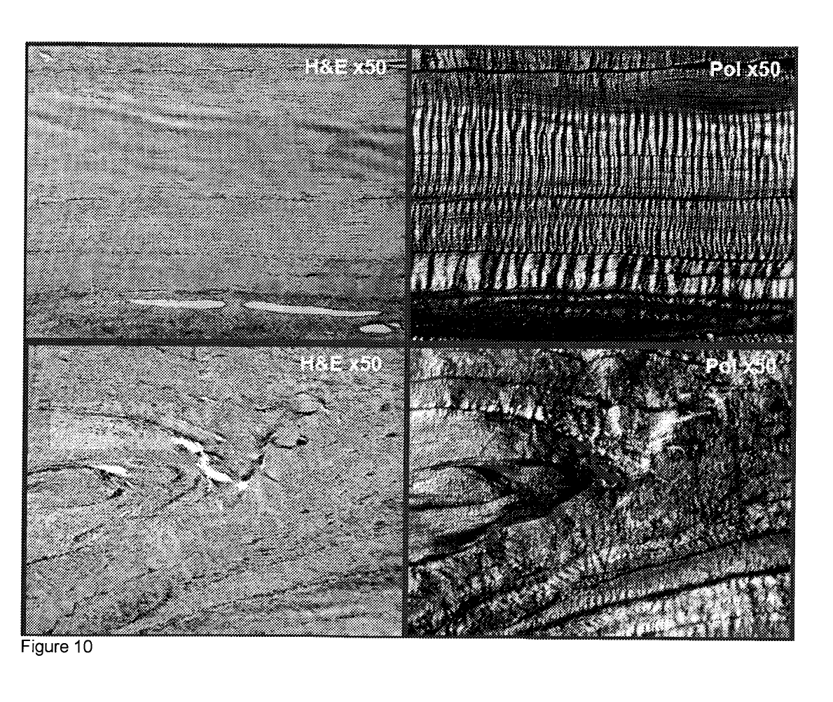

FIG. 10 is a set of photomicrographs of histological sections stained with hematoxylin and eosin (left images) and visualized with polarized light (right side), showing the very different tendon architecture of experimental tendon (upper images) versus control tendon (lower images). Histological specimens were scored on a scale ranging from 11 for normal to 44 for severe damage, based on the following characteristics: tendon cell linearlity (1=linear to 4=all rounded), tendon cell density (1=sparse to 4=sheets of cells), free hemorrhage (1=none to 4=predominantly hemorrhage), neovasculature (1=normal endotenal to 4=severely increased), inflammatory cell infiltrate (1=none to 4=severely increased), collagen fiber linearity (1=linear to 4=no linear areas), collagen fiber uniformity (1=uniform diameter to 4=disarray), collagen fiber crimping under polarized light (1=coarse even to 4=no crimp), epitenal thickening (1=normal 1-2 cells to 4=massive thickening), collagen type I deposition (1=>90% to 4=<10%), and collagen type II deposition (1=<10% to 4=>90%). The histologic scores are summarized in the following table.

| | Experimental | Control | |
|---|---|---|---|
| | Mean (SD) | Mean (SD) | P Value |
| Tendon cell shape | 1.13 (0.35) | 2.06 (0.32) | 0.008 |
| Tendon cell density | 2.00 (0.0) | 2.63 (0.52) | 0.031 |
| Free hemorrhage | 1.31 (0.37) | 1.69 (0.70) | 0.188 |
| Neovascularization | 1.81 (0.46) | 1.75 (0.38) | 0.406 |
| Inflammatory cell infiltrate | 1.56 (0.86) | 1.13 (0.23) | 0.156 |
| Tendon fiber linearity | 1.69 (0.46) | 2.69 (0.26) | 0.004 |
| Tendon fiber uniformity | 1.75 (0.38) | 2.94 (0.18) | 0.004 |
| Tendon polarized crimp | 1.63 (0.44) | 2.97 (0.34) | 0.004 |
| Epitenon thickness | 2.31 (0.37) | 3.06 (0.42) | 0.008 |
| Total score | 15.38 (1.69) | 21.13 (1.81) | 0.004 |

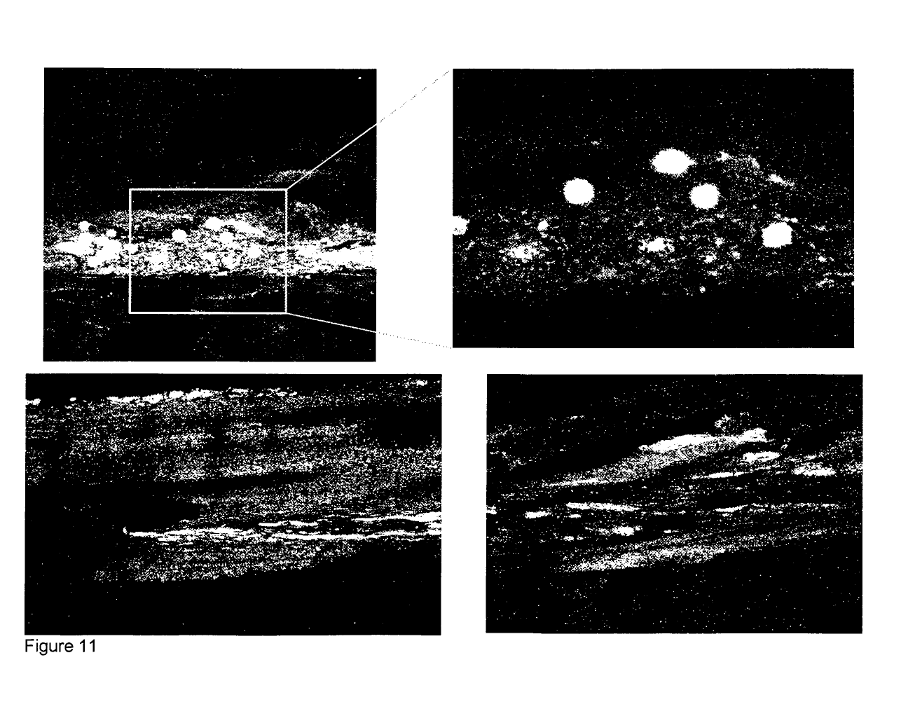

Survival of transplanted cells was confirmed by fluorescence in situ hybridization to detect the presence of the Y chromosome in male cells transplanted into a female host. As shown in FIG. 11, at two weeks post injection (upper images), a number of rounded cells are positive for the Y chromosome. The boxed area in the upper left image is shown, enlarged, in the upper right image. By 8 weeks (lower images), the Y-positive cells are no longer rounded, but have become aligned fibers.

The results of this controlled study confirm that pluripotent stem cells transplanted in accordance with the invention into damaged connective tissue effect significant and remarkable healing, observable at the histological, ultrasonographic, and MRI levels via blind analysis. Moreover, this healing occurs without significant adverse reactions.

### Example 12

### Treatment of Severe Arthritis and Cruciate Ligament Injuries in Canines

Initial research described in the Examples above has shown the potential of pluripotent cells of the invention as an effective and safe regenerative approach; first in equine musculoskeletal injuries, and more recently in canine injuries—along with an optimal dose of 30,000 cells per pound of body weight for intra-venous injection and 500 thousand cells/2.0 ml for intra-articular injection. In particular, these stem cells are pluripotent and non-immunogenic, which gives them the capability to generate almost any type of cells without the danger of immune mediated rejection.

### Stem Cell Procurement

Canine fetuses were obtained at 26 days post gestation by flushing. Sample was then washed multiple times in HBSS supplemented with Gentamycin (MP Biomedicals 1676045, Solon, Ohio) and Fungizone (Omega Scientific FG-70, Tarzana, Calif.). The fetal tissue (specifically brain, spinal cord, liver and heart) was dissected under sterile conditions in Hanks Balanced Salt Solution (HBSS, Invitrogen 14025, Carlsbad Calif.) supplemented with Gentamycin and Fungizone. After multiple washes (at least ten times in anti-microbial and anti-fungal HBSS), each organ was separately minced with microscissors under a dissecting microscope (Olympus SZ61, Center Valley, Pa.) in a laminar flow hood (1846, Thermo Scientific, Waltham, Mass.) and then triturated with sterile fire-polished Pasteur pipettes until a single cell suspension was obtained.

Cell counts were performed using a hemocytometer, and viability was tested using Trypan Blue exclusion and documented in laboratory notebooks. Each tissue sample produced approximately 40 million cells. These cells were explanted into culture flasks at a final concentration of ˜10 million cells/flask, in culture medium containing the following components:

EMEM (Eagle's Essential Media, Lonza RR116254, Walkersville MD)
B27 (Invitrogen 17504, Carlsbad, CA)
Calcium Chloride (Fisher Scientific, Pittsburgh, PA)
Epidermal Growth Factor (Peprotech 100-15, Rocky Hill, NJ)
Basic Fibroblast Growth Factor (Peprotech 100-18B, Rocky Hill, NJ)
Transforming Growth Factor Alpha (Peprotech 100-16A, Rocky Hill, NJ)
Leukemia Inhibitory Factor (Millipore LIF1010, Temecula, CA)
Glutamax (Invitrogen 25030, Carlsbad, CA)
N2 Supplement (Invitrogen 17502, Carlsbad, CA)

Cells were cultured in an incubator (Thermo Scientific, Fisher) for one week to determine that there was no contamination, as defined by examination under a light microscope. Samples of the cell culture were sent to an outside laboratory for microbial and viral screening (Bionique Testing Laboratories, Saranac Lake, Calif.). If results are negative, cell cultures remain in the manufacturing process.

### Stem Cell Culture and Immunostaining

Once sterility of cell culture was confirmed, cells were explanted into culture flasks at a final concentration of 400,000 cells/flask in the above culture medium. Once a week, cells were centrifuged, supernatant was discarded except 2 ml, in which cells were resuspended, transferred to the culture flask and 4 ml of fresh culture medium were added. Four days later, 4 mL of fresh culture medium was added to each flask. This cycle was repeated weekly. Cells were passaged 4-5 times before immunohistochemical testing for homogeneity and pluripotency.

To assess homogeneity and pluripotency, a 1.7 mL sample of cells were taken and centrifuged using the Centra CL2 Cytospin (Thermo Fisher IEC, Waltham, Mass.) to obtain a monolayer of cells on a microscope slide. Slides were fixed with 4% formaldehyde for 20 minutes, and permeabilized for one hour with 0.1% Triton-X. Cells were incubated with primary antibodies for anti-rabbit Oct-4 (1:200, Millipore AB3209), anti-mouse Major Histocompatibility Complex (MHC, 1:50, Invitrogen MM3415) anti-rabbit Nestin (1:250, Abcam AB5968), anti-rabbit Telomerase (1:200, Calbiochem 582005), and anti-mouse Ki-67 (1:50, Invitrogen 18-0192Z) overnight at room temperature. Anti-mouse (1:100, Vector BA-2001) or anti-rabbit secondary antibody (1:100, Vector BA-1000) was applied for two hours at room temperature. This was followed by Avidin-Biotin Kit (Vector PK-6100) for 30 minutes, and DAB complex (Dako K3468) for one minute.

Canine cells studied immunohistologically for marker expression after 2 months in culture were found to be 80% positive for Oct4, 45% positive for nestin, 60% positive for Nanog, 15% positive for Ki-67 (indicator of cell growth and division), 90% for telomerase (indicator of sternness and ability to divide without aging), and 0% positive for MHC.

Cell cultures expressing markers for pluripotency (Oct-4, Nestin, Telomerase) and not expressing any Major Histocompatibility Complex (MHC), are considered to be suitable for transplant.

### Cell Preparation for Transplant

To prepare “OK200” for transplant, cells are counted using a hemocytometer, and the appropriate dose of cells is condensed into 2 mL culture media. Cells are packaged in a 2 mL cryovial and transported on ice to transplant site.

### Laboratory Studies

To determine that embryonic stem cells are functioning cells and present as healing tissue, the inventor and staff have performed more than 400 experiments in rodents. These experiments have proven that our cells migrate to the site of the problem, differentiate according to the nature of the problem, and restore the structure and function. Documentation is available in the form of numerous histological and immunohistochemical slides, and videotaped evidence of behavioral recovery. Further, our experiments have tracked embryonic stem cells using antihuman antibodies, and have consistently shown that they migrate to the site of injury and differentiate according to the nature of the injury.

Allogeneic pluripotent cell transplantation can be used for treatment of chronic osteoarthritis, ligamentous and tendon injuries, including canine hip dysplasia, canine elbow dysplasia, cranial cruciate ligament injury, and achilles tendon injury.

Optionally, treated dogs are followed for four months post transplantation, monitoring the following end points:
Improved soundness of healing using a standardized orthopedic examination rating score by attending veterinarian(s) on the following parameters: (1) Lameness at walk and trot (six point scale); (2) Pain on manipulation (3 point scale); (3) Range of motion (4 point scale); and (4) Functional disability (5 point scale).
Baseline and improvements in follow up ratings (30, 60, and 120 days) by both veterinarians and owners on a numeric rating scale: Cincinnati Orthopedic Disability Index (CODI)—13 parameters; and lack of adverse events to injection of transplanted cells.

### Treatment Procedure for Osteoarthritis

Subjects are pretreated with dexamethasone, 6 mg IV, and diphenhydramine, 40 mg IV. Both treatments are given 35 minutes prior to stem cell injection. The treatment consists of 30,000 stem cells per pound in 50 mls Lactate Ringer Solution (LRS) LRS, administered by IV slowly over 12 minutes.

### Treatment for Cruciate Ligament Injuries

Subjects are treated by intra-articular or ligamentous injection. The animal is first sedated (general anesthesia may be used if necessary). Next the dog is pre-medicated with 0.25-0.5 mg./lb of Dexamethasone intravenously (administer at least 30 minutes before stem cell procedure). The area to be injected in surgically prepared. Using sterile surgical technique, the required amount of Embryonic Stem Cells are injected through a 21 or 25 gauge needle. The animal is then covered with antibiotics for 10-14 days.

From the foregoing it will be appreciated that, although specific embodiments of the invention have been described herein for purposes of illustration, various modifications may be made without deviating from the spirit and scope of the invention. Accordingly, the invention is not limited except as by the appended claims.

What is claimed is:

1. A method of ameliorating connective tissue damage in a mammalian subject, the method comprising introducing into a site of connective tissue damage in the subject about 1 to about 10 million stem cells, wherein the stem cells are derived from fetal mammalian brain.

2. The method of claim 1, wherein the stem cells do not express major histocompatibility complex (MHC).

3. The method of claim 1, wherein the stem cells are allogeneic.

4. The method of claim 1, wherein the stem cells are cultured for about 30 days prior to the introducing.

5. The method of claim 4 wherein the stem cells are immunopositive for telomerase, Oct-4, SSEA-4 and Nanog, and do not express major histocompatibility complex (MHC) or p53.

6. The method of claim 4 wherein the stem cells are cultured in a medium having a total calcium concentration of 0.03 to 0.15 mM and comprising:
(a) about 15-100 ng/ml epidermal growth factor (EGF);
(b) about 10-150 ng/ml basic fibroblast growth factor (bFGF);
(c) about 10-75 ng/ml transforming growth factor-alpha (TGFα); and
(d) about 30-50 ng/ml insulin-like growth factor (IGF).

7. The method of claim 6, wherein the medium further comprises:
(e) about 1-3% by volume B27;
(f) about 40-60 ng/ml leukemia inhibitory factor (LIF);
(g) about 0.05-0.2 mM GLUTAMAX; and
(h) about 0.5-2% by volume N2 supplement.

8. The method of claim 7, wherein the medium is Eagle's minimum essential medium (EMEM) and comprises:
(a) about 40 ng/ml epidermal growth factor (EGF);
(b) about 40 ng/ml basic fibroblast growth factor (bFGF); and
(c) about 40 ng/ml transforming growth factor-alpha (TGFα).
(d) about 40 ng/ml insulin-like growth factor (IGF);
(e) about 50 ng/ml leukemia inhibitory factor (LIF);
(f) about 2% by volume B27;
(g) about 0.05-0.2 mM GLUTAMAX;
(h) about 0.5-2% by volume N2 supplement; and
(i) about 0.05 mM calcium chloride.

9. The method of claim 1, wherein the introducing comprises injection into the site of connective tissue damage.

10. The method of claim 9, wherein the injection is performed under ultrasound guidance.

11. The method of claim 1, wherein the cells are derived from equine fetal tissue.

12. The method of claim 1, wherein the mammalian subject is equine.

13. The method of claim 1, wherein the mammalian subject is canine or feline.

14. The method of claim 1, wherein the mammalian subject is human.

15. The method of claim 1, wherein the connective tissue damage comprises ligament damage, osteochondrosis, tendonitis, navicular syndrome, cartilage damage, laminitis or arthritis.

16. The method of claim 1, wherein the introducing comprises intravenous injection.

17. The method of claim 1, wherein the introducing comprises distal end perfusion.

18. A kit comprising:
(a) a container comprising about 1 to about 10 million stem cells, wherein the stem cells are derived from fetal mammalian brain;
(b) a label that indicates use of the cells for implantation into a site of connective tissue damage.

19. The kit of claim 18, further comprising:
(c) a needle suitable for transcutaneous injection.

20. The kit of claim 18, wherein the container comprises a syringe.

21. The kit of claim 18, wherein the fetal mammalian brain is equine.

22. The kit of claim 18, wherein the cells do not express MHC.

23. The kit of claim 18, further comprising a second container, the second container comprising platelet-rich plasma.
